SEQUENCE LISTING (93 sequences)

SEQ ID NO: 1 - DNA, 69 bp, Homo sapiens
atggatgcaatgaagagagggctctgctgtgtgctgctgctgtgtggagcagtcttcgtttcgcccagc

SEQ ID NO: 2 - DNA, 17 bp, Artificial Sequence
  Synthetic Oligonucleotide Primer
ccgccatgttgacattg

SEQ ID NO: 3 - DNA, 64 bp, Artificial Sequence
  Synthetic Oligonucleotide Primer
ggcagatctgggaggctagcgtaatccggaacatcgtatgggtacattgttaagtcgacggtgc

SEQ ID NO: 4 - DNA, 56 bp, Artificial Sequence
  Synthetic Oligonucleotide Primer
gatcccgggtacccatacgatgttccggattacgcttaggggagatctcagacatg

SEQ ID NO: 5 - DNA, 32 bp, Artificial Sequence
  Synthetic Oligonucleotide Primer
caggatatcatgcctgcaggacgactctagag

SEQ ID NO: 6 - DNA, 169 bp, Artificial Sequence
  Synthetic Oligonucleotide Primer
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatatacc

SEQ ID NO: 7 - DNA, 238 bp, Artificial Sequence
  Synthetic Oligonucleotide Primer
ggggggggttctcatcatcatcatcatcattaataaaagggcgaattccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

SEQ ID NO: 8 - DNA, 35 bp, Artificial Sequence
  Synthetic Oligonucleotide Primer
gaaggagatataccatgcatcatcatcatcatcat

SEQ ID NO: 9 - DNA, 20 bp, Artificial Sequence
  Synthetic Oligonucleotide Primer
tgatgatgagaacccccccc

SEQ ID NO: 10 - DNA, 50 bp, Artificial Sequence
  Synthetic Oligonucleotide Primer
gaaggagatataccatgcatcatcatcatcatcatatgtctaagatctat

SEQ ID NO: 11 - DNA, 40 bp, Artificial Sequence
  Synthetic Oligonucleotide Primer
tgatgatgagaacccccccctcagaatctaatgatgacgt

SEQ ID NO: 12 - DNA, 50 bp, Artificial Sequence
  Synthetic Oligonucleotide Primer
gaaggagatataccatgcatcatcatcatcatcatatggacatgatgctt

SEQ ID NO: 13 - DNA, 40 bp, Artificial Sequence
  Synthetic Oligonucleotide Primer
tgatgatgagaaccccccccttagttcacggaaatatcgc

SEQ ID NO: 14 - DNA, 50 bp, Artificial Sequence
  Synthetic Oligonucleotide Primer
gaaggagatataccatgcatcatcatcatcatcatatgaatttacagaaa

SEQ ID NO: 15 - DNA, 40 bp, Artificial Sequence
  Synthetic Oligonucleotide Primer
tgatgatgagaaccccccccctacttgtacggtagcgacg

SEQ ID NO: 16 - DNA, 50 bp, Artificial Sequence
  Synthetic Oligonucleotide Primer
gaaggagatataccatgcatcatcatcatcatcatatgttcaacatgaat

SEQ ID NO: 17 - DNA, 40 bp, Artificial Sequence
  Synthetic Oligonucleotide Primer
tgatgatgagaaccccccccttatctaagtcctgttgatc

SEQ ID NO: 18 - DNA, 50 bp, Artificial Sequence
  Synthetic Oligonucleotide Primer
gaaggagatataccatgcatcatcatcatcatcatatgacgaccgtacca

SEQ ID NO: 19 - DNA, 40 bp, Artificial Sequence
  Synthetic Oligonucleotide Primer
tgatgatgagaaccccccccttaaataattttaattcgtt

SEQ ID NO: 20 - DNA, 50 bp, Artificial Sequence
  Synthetic Oligonucleotide Primer
gaaggagatataccatgcatcatcatcatcatcatatggacggaactctt

SEQ ID NO: 21 - DNA, 40 bp, Artificial Sequence
  Synthetic Oligonucleotide Primer
tgatgatgagaaccccccccttactcatatggacgccgtc

SEQ ID NO: 22 - DNA, 50 bp, Artificial Sequence
  Synthetic Oligonucleotide Primer
gaaggagatataccatgcatcatcatcatcatcatatgggaactgctgca

SEQ ID NO: 23 - DNA, 40 bp, Artificial Sequence
  Synthetic Oligonucleotide Primer
tgatgatgagaaccccccccttatatatttagtttatctt

SEQ ID NO: 24 - DNA, 50 bp, Artificial Sequence
  Synthetic Oligonucleotide Primer
gaaggagatataccatgcatcatcatcatcatcatatggcggcggtgaaa

SEQ ID NO: 25 - DNA, 40 bp, Artificial Sequence
  Synthetic Oligonucleotide Primer
tgatgatgagaaccccccccttagataaatgcggtaacga

SEQ ID NO: 26 - DNA, 50 bp, Artificial Sequence
  Synthetic Oligonucleotide Primer
gaaggagatataccatgcatcatcatcatcatcatatgaatacccgtacc

SEQ ID NO: 27 - DNA, 40 bp, Artificial Sequence
  Synthetic Oligonucleotide Primer
tgatgatgagaaccccccccttatactataatagtactag

SEQ ID NO: 28 - DNA, 50 bp, Artificial Sequence
  Synthetic Oligonucleotide Primer
gaaggagatataccatgcatcatcatcatcatcatatgcatgtgcctgcc

SEQ ID NO: 29 - DNA, 40 bp, Artificial Sequence
  Synthetic Oligonucleotide Primer
tgatgatgagaacccccccctcagacacacgctttgagtt

SEQ ID NO: 30 - DNA, 50 bp, Artificial Sequence
  Synthetic Oligonucleotide Primer
gaaggagatataccatgcatcatcatcatcatcatatggccgaggaattt

SEQ ID NO: 31 - DNA, 40 bp, Artificial Sequence
  Synthetic Oligonucleotide Primer
tgatgatgagaacccccccctcaacaagttctcaataccc

SEQ ID NO: 32 - DNA, 50 bp, Artificial Sequence
  Synthetic Oligonucleotide Primer
gaaggagatataccatgcatcatcatcatcatcatatgaattctcatttt

SEQ ID NO: 33 - DNA, 40 bp, Artificial Sequence
  Synthetic Oligonucleotide Primer
tgatgatgagaaccccccccttagttcaaaagtctcgacg

SEQ ID NO: 34 - DNA, 50 bp, Artificial Sequence
  Synthetic Oligonucleotide Primer
gaaggagatataccatgcatcatcatcatcatcatatggatacagatgtt

SEQ ID NO: 35 - DNA, 40 bp, Artificial Sequence
  Synthetic Oligonucleotide Primer
tgatgatgagaaccccccccctatattcctacatcgtgac

SEQ ID NO: 36 - DNA, 50 bp, Artificial Sequence
  Synthetic Oligonucleotide Primer
gaaggagatataccatgcatcatcatcatcatcatatgataagtaattac

SEQ ID NO: 37 - DNA, 40 bp, Artificial Sequence
  Synthetic Oligonucleotide Primer
tgatgatgagaaccccccccttaaatcgccgaatgaacaa

SEQ ID NO: 38 - DNA, 50 bp, Artificial Sequence
  Synthetic Oligonucleotide Primer
gaaggagatataccatgcatcatcatcatcatcatatgagtaaggtaatc

SEQ ID NO: 39 - DNA, 40 bp, Artificial Sequence
  Synthetic Oligonucleotide Primer
tgatgatgagaaccccccccttatacattgaatattggct

SEQ ID NO: 40 - DNA, 50 bp, Artificial Sequence
  Synthetic Oligonucleotide Primer
gaaggagatataccatgcatcatcatcatcatcatatggctgaatggcat

SEQ ID NO: 41 - DNA, 40 bp, Artificial Sequence
  Synthetic Oligonucleotide Primer
tgatgatgagaaccccccccttaattaccagttgcacgta

SEQ ID NO: 42 - DNA, 50 bp, Artificial Sequence
  Synthetic Oligonucleotide Primer
gaaggagatataccatgcatcatcatcatcatcatatgaagaacgtattg

SEQ ID NO: 43 - DNA, 40 bp, Artificial Sequence
  Synthetic Oligonucleotide Primer
tgatgatgagaaccccccccttattcggtaacaggtggcc

SEQ ID NO: 44 - DNA, 50 bp, Artificial Sequence
  Synthetic Oligonucleotide Primer
gaaggagatataccatgcatcatcatcatcatcatatgcgtgcacttttt

SEQ ID NO: 45 - DNA, 40 bp, Artificial Sequence
  Synthetic Oligonucleotide Primer
tgatgatgagaacccccccctcatccatccgtagatgatt

SEQ ID NO: 46 - DNA, 50 bp, Artificial Sequence
  Synthetic Oligonucleotide Primer
gaaggagatataccatgcatcatcatcatcatcatatggatctgtcacga

SEQ ID NO: 47 - DNA, 40 bp, Artificial Sequence
  Synthetic Oligonucleotide Primer
tgatgatgagaaccccccccttagtttttctttacacaat

SEQ ID NO: 48 - DNA, 50 bp, Artificial Sequence
  Synthetic Oligonucleotide Primer
gaaggagatataccatgcatcatcatcatcatcatatggcgatgttttac

SEQ ID NO: 49 - DNA, 40 bp, Artificial Sequence
  Synthetic Oligonucleotide Primer
tgatgatgagaaccccccccttacctcgatggtgccgccg

SEQ ID NO: 50 - DNA, 50 bp, Artificial Sequence
  Synthetic Oligonucleotide Primer
gaaggagatataccatgcatcatcatcatcatcatatgacggccaacttt

SEQ ID NO: 51 - DNA, 40 bp, Artificial Sequence
  Synthetic Oligonucleotide Primer
tgatgatgagaacccccccctcaattcatacgccggaata

SEQ ID NO: 52 - DNA, 573 bp, Vaccinia virus
atgtctaagatctatattgacgagcgttctgacgcagagattgtgtgtgcggctattaaaaacattggaatcgaaggagctactgctgcacaactaactagacaacttaatatggagaagcgagaagttaataaagctctgtacgatcttcaacgtagtgctatggtgtacagctccgacgatattcctcctcgttggtttatgacaacggaggcggataagccggatgctgatgttatggctgacgccataatagatgatgtatcccgcgaaaaatcaatgagagaggatcataagtcttttgatgatgttattccggctaaaaaaattattgattggaaagatgctaaccctgtcaccattattaatgagtactgccaaataactaagagagattggtcttttcgtattgaatcagttgggcctagtaactctcctacattttatgcctgtgtagatatcgacggaagagtattcgataaggccgatggaaaatctaaacgagatgctaaaaataatgcagctaaattggctgtagataaacttcttgggtacgtcatcattagattctga

SEQ ID NO: 53 - DNA, 273 bp, Vaccinia virus
atggacatgatgcttatgattggaaattatttttccggcgtgctaatcgctggaatcattcttttgattctttcgtgtatcttcgcctttattgactttagtaagtctaccagtcccactcgtacatggaaagtattgagtattatggcgtttatacttggtattattatcacagtcggaatgctaatttattctatgtggggaaagcactgcgcgccccacagagttagcggagtcattcacaccaatcatagcgatatttccgtgaactaa

SEQ ID NO: 54 - DNA, 156 bp, Vaccinia virus
atgaatttacagaaattatctctggctatatatcttactgcgacatgttcgtggtgttatgaaacatgcataagaaaaactgcgttgtatcatgacattcaattggagcatgtagaagacaataaagatagtgtagcgtcgctaccgtacaagtag

SEQ ID NO: 55 - DNA, 444 bp, Vaccinia virus
atgttcaacatgaatattaactcaccagttagatttgttaaggaaactaacagagctaaatctcctactaggcaatcgccgggtgctgccggatatgatttgtatagcgcttacgattatactatccctccaggagaaagacagttaattaagacagatattagtatgtccatgcctaagatttgctatggtagaatagctcctaggtctggtctgtcactaaaaggcattgatataggaggtggtgtaatagacgaagattataggggaaacataggagtcattcttattaataatggaaaatgtacgtttaatgtaaatactggagatagaatagctcagctaatctatcaacgtatatattatccagaactggaagaagtacaatctctagatagtacaaatagaggagatcaagggtttggatcaacaggacttagataa

SEQ ID NO: 56 - DNA, 957 bp, Vaccinia virus
atgacgaccgtaccagtgacggatatacaaaacgatttaattacagagttttcagaagataattatccatctaacaaaaattatgaaataactcttcgtcaaatgtctattctaactcacgttaacaacgtggtagatagagaacataatgccgccgtagtgtcatctccagaggaaatatcctcacaacttaatgaagatctatttccagatgatgattcaccggccactattatcgaacgagtacaacctcatactactattattgacgatactccacctcctacgtttcgtagagagttattgatatcggaacaacgtcaacaacgagaaaaaagatttaatattacagtatcgaaaaatgctgaagcaataatggaatctagatctatgataacttctatgccaacacaaacaccatccttgggagtagtttatgataaagataaaagaattcagatgttagaggatgaagtggttaatcttagaaatcaacgatctaatacaaaatcatctgataatttagataattttaccaaaatactatttggtaagactccgtacaaatcaacagaagttaataagcgtatagccatcgttaattatgcaaatttgaacgggtctcccttatcagtcgaggacttggatgtttgttcagaggatgaaatagatagaatctataaaacgattaaacaatatcacgaaagtagaaaacgaaaaattatcgtcactaacgtgattattattgtcataaacattatcgagcaggcattgctaaaactcggatttgaagaaatcaaaggactgagtaccgatatcacttcagaaattatcgatgtggagatcggagatgactgcgatgctgtagcatctaaactaggaatcggtaacagtccggttcttaatattgtattgtttatactcaagatattcgttaaacgaattaaaattatttaa

SEQ ID NO: 57 - DNA, 333 bp, Vaccinia virus
atggacggaactcttttccccggagatgacgatcttgcaattccagcaactgaatttttttctacaaaggctgctaaaaagccagaggctaaacgcgaagcaattgttaaagccgatgaagacgacaatgaggaaactctcaaacaacggctaactaatttggaaaaaaagattactaatgtaacaacaaagtttgaacaaatagaaaagtgttgtaaacgcaacgatgaagttctatttaggttggaaaatcacgctgaaactctaagagcggctatgatatctctggctaaaaagattgatgttcagactggacggcgtccatatgagtaa

SEQ ID NO: 58 - DNA, 501 bp, Vaccinia virus
atgggaactgctgcaacaattcagactcccaccaaattaatgaataaagaaaatgcagaaatgattttggaaaaaattgttgatcatatagttatgtatattagtgacgaatcaagtgattcagaaaataatcctgaatatattgattttcgtaacagatacgaagactatagatctctcattataaaaagtgatcacgagtttgtaaagctatgtaaaaatcatgcggagaaaagttctccagaaacgcaacaaatgattatcaaacacatatacgaacaatatcttattccagtatctgaagtactattaaaacctataatgtccatgggtgacataattacatataacggatgtaaagacaatgaatggatgctagaacaactctctaccctaaactttaacaatctccgcacatggaactcatgtagcataggcaatgtaacgcgtctgttttatacattttttagttatctgatgaaagataaactaaatatataa

SEQ ID NO: 59 - DNA, 975 bp, Vaccinia virus
atggcggcggtgaaaactcctgttattgttgtgccagttattgatagacctccatcagaaacatttcctaatgttcatgagcatattaatgatcagaagttcgatgatgtaaaggacaacgaagttatgccagaaaaaagaaatgttgtggtagtcaaggatgatccagatcattacaaggattatgcgtttatacagtggactggaggaaacattagaaatgatgacaagtatactcacttcttttcagggttttgtaacactatgtgtacagaggaaacgaaaagaaatatcgctagacatttagccctatgggattctaatttttttaccgagttagaaaataaaaaggtagaatatgtagttattgtagaaaacgataacgttattgaggatattacgtttcttcgtcccgtcttgaaggcaatgcatgacaaaaaaatagatatcctacagatgagagaaattattacaggcaataaagttaaaaccgagcttgtaatggacaaaaatcatgccatattcacatatacaggagggtatgatgttagcttatcagcctatattattagagttactacggcgctgaacatcgtagatgaaattataaagtctggaggtctatcatcgggattttattttgaaatagccagaatcgaaaacgaaatgaagatcaataggcagatactggataacgccgccaaatatgtagaacacgatccccgacttgttgcagaacaccgcttcgaaaacatgaaaccgaatttttggtctagaataggaacggcagctgctaaacgttatccaggagttatgtacgcgtttactactccactgatttcattttttggattgtttgatattaatgttataggtttgattgtaattttgtttattatgtttatgctcatctttaacgttaaatctaagctgttatggttccttacaggaacattcgttaccgcatttatctaa

SEQ ID NO: 60 - DNA, 1053 bp, Vaccinia virus
atgaatacccgtaccgatgttacaaacgataatatagacaaaaatccaaccaaacgaggtgatagaaatataccaggaagaaatgaaagatttaatgaccaaaatagattcaacaacgatataccaaagcctaaaccaagactacagcctaatcagccaccgaaacaagataataaatgcagagaagagaatggagattttatcaatattagattgtgtgcctacgagaaggaatattgcaatgacggatatctatctcctgcctattatatgttaaaacaggtggatgatgaagaaatgagttgctggtcagaactatcgtcgttggtgagatccagaaaggcggtgggatttcctctattaaaggcggctaaacgtatttctcatggatcgatgctatattttgaacagttcaaaaacagtaaagttgtgaaattaaccccgcaagttaaatgtttaaatgatactgttatttttcaaactgtagttattttatattccatgtataaacgtggcatatattctaacgaattttgttttaatctggtttctattcccagaacgaacattgttttttctgttaatcaattaatgtttaacatttgtacagacatattggtagttctatctatttgcggcaaccggctctatagaacaaatctaccacagtcgtgttacttaaatttcatacacggccatgagacaatagcccgtagaggatatgaacactccaattactttttcgagtggttgataaaaaatcacatatcgctattgaccaagcaaacgatggatattctcaaggtaaagaaaaagtatgctacaggagcaccagtaaataggttgttagaacctggtacactggtatatgtgcccaaagaagattattactttataggcatatcactcaccgatgtgtcaattagcgataatgtcagagtattattttccacagatggaatagtgttagaaatagaagactttaatatcaagcatttatttatggcaggtgagatgtttgttagaagtcagtctagtactattatagtataa

SEQ ID NO: 61 - DNA, 726 bp, Vaccinia virus
atgcatgtgcctgccagtcttcagcaatcctcctcctcgtgtacggaagaagaaaacaaacatcatatgggaatcgatgttattatcaaagtcacaaagcaagaccaaacaccgaccaatgataagatttgccaatccgtaacggaaattacagagtccgagtcagatccagatcccgaggtggaatcagaagatgattccacatcagtcgaggatgtagatcctcctaccacttattactccatcatcggtggaggtctgagaatgaactttggattcaccaaatgtcctcagattaaatccatctcagaatccgctgatggaaacacagtgaatgctagattgtccagcgtgtccccaggacaaggtaaggactctcccgcgatcactcatgaagaagctcttgctatgatcaaagactgtgaggtgtctatcgacatcagatgtagcgaagaagagaaagacagcgacatcaagacccatccagtactcgggtctaacatctctcataagaaagtgagttacgaagatatcatcggttcaacgatcgtcgatacaaaatgcgtcaagaatctagagtttagcgttcgtatcggagacatgtgcaaggaatcatctgaacttgaggtcaaggatggattcaagtatgtcgacggatcggcatctgaaggtgcaaccgatgatacttcactcatcgattcaacaaaactcaaagcgtgtgtctga

SEQ ID NO: 62 - DNA, 327 bp, Vaccinia virus
atggccgaggaatttgtacaacaaaggttggccaataacaaagtgacaatttttgtcaagtatacatgtcctttttgtagaaatgcactggatattctaaataagtttagtttcaaaagaggagcgtatgaaattgtcgatattaaagaatttaaacccgaaaatgaattgcgggattattttgaacaaattactggtggtagaactgttcctagaatcttttttgggaaaacttctattggtggatatagcgacctgttggaaatagacaacatggacgcattgggtgatattctatcatctattggggtattgagaacttgttga

SEQ ID NO: 63 - DNA, 306 bp, Vaccinia virus
atgaattctcattttgcatctgctcatactccgttttatatcaataccaaagaaggaagatatctggttctaaaagccgttaaagtatgcgatgttagaactgtagaatgcgaaggaagtaaagcttcctgcgtactcaaagtagataaaccctcatcacccgcgtgtgagagaagaccttcgtccccgtccagatgcgagagaatgaataaccctggaaaacaagtcccgtttatgagaacggacatgctacaaaatatgttcgcggctaatcgcgacaacgtggcgtcgagacttttgaactaa

SEQ ID NO: 64 - DNA, 219 bp, Vaccinia virus
atggatacagatgttacaaatgtagaagatatcataaatgaaatagatagagagaaagaagaaatactaaaaaatgtagaaattgaaaataataaaaacattaacaagaatcatcccaatgaatatattagagaagcactcgttattaataccagtagtaatagtgattccattgataaagaagttatagaatgtatctgtcacgatgtaggaatatag

SEQ ID NO: 65 - DNA, 162 bp, Vaccinia virus
atgataagtaattacgagccgttgctgctgttagttataacatgctgtgtactactatttaattttaccatatcttcgaaaacaaaaatagatattatttttgcagtacaaactattgtttttatatggtttatattccactttgttcattcggcgatttaa

SEQ ID NO: 66 - DNA, 810 bp, Vaccinia virus
atgagtaaggtaatcaagaagagagttgaaacttcaccaagacctactgcatctagcgattctctacagacttgtgcgggtgttatagagtatgcaaaatcgattagtaaatctaatgcaaaatgtatcgaatacgttacactaaatgcttctcaatacgctaattgttcgtctatctctataaaacttactgatagtttatctagtcaaatgacttccacttttattatgttggaaggagagactaaactttataaaaataaatctaaacaagatagaagcgatggatactttctaaaaataaaagttaccgcggctagtcctatgttgtatcaacttctagaagccgtctatggaaacattaagcacaaggaacgcattccaaattctttgcatagtctttcggtggaaactattacagagaaaacatttaaggatgaatccatcttcatcaacaaattaaacggatccatggtagaatatgtttcgactggagaatcatccattctcagatctatagaaggtgaactagaatcactcagtaaaagagaaagacaattggccaaggcaattatcacacctgtagttttctatagatccggaacggaaacaaaaattacattcgcactcaagaaactaatcattgatagagaagtggtggctaacgttatcggactctctggagatagtgaacgtgtatcaatgactgaaaatgtagaagaagatctggctcgtaatctgggacttgttgatattgatgatgaatatgatgaagatagcgataaagaaaagccaatattcaatgtataa

SEQ ID NO: 67 - DNA, 402 bp, Vaccinia virus
atggctgaatggcataaaattatcgaggatatctcaaaaaataataagttcgaggatgccgccatcgttgattacaagactacaaagaatgttctagctgctattcctaacagaacatttgccaagattaatccgggtgaaattattcctctcatcactaatcgtaatattctaaaacctcttattggtcagaaatattgtattgtatatactaactctctaatggacgagaacacgtatgctatggagttgcttactgggtacgcccctgtatctccgatcgttatagcgagaactcataccgcacttatatttttgatgggtaagccaacaacatccagacgtgacgtgtatagaacgtgtagagatcacgctacccgtgtacgtgcaactggtaattaa

SEQ ID NO: 68 - DNA, 375 bp, Vaccinia virus
atgaagaacgtattgattattttcggtaaaccgtattgtagtatttgtgaaaatgtcagtgagtcagtagaagaattaaaatccgagtatgatatactccatgttgatatcttatcattttttttaaaggatggtgattcaagtatgctgggtgacgtaaagcgcggaaccctaataggaaactttgcagcgcatctatctaactacatcgtttccattttcaaatacaatccacagacaaaacaaatggcatttgtggacattaataaatccttggatttcaccaaaaccgataaatcgctggtgaatttggaaattctaaaatccgaaatagaaaaagctacctatggagtttggccacctgttaccgaataa

SEQ ID NO: 69 - DNA, 945 bp, Vaccinia virus
atgcgtgcacttttttataaagatggtaaactctttaccgataataattttttaaatcctgtattagatgataatccagcgtatgaggttttgcaacatgttaaaattcctactcatttaacagatgtagtagtatatgaacaaacgtgggaagaggcattaactagattaatttttgtgggaagcgattcaaaaggacgtagacaatacttttacggaaaaatgcatgtacagaatcgcaacgctaaaagagatcgtatttttgttagagtatataacgttatgaaacgaattaattgttttataaacaaaaatataaagaaatcgtccacagattccaattatcagttggcggtttttatgttaatggaaactatgttttttattagatttggtaaaatgaaatatcttaaggagaatgaaacagtagggttattaacactaaaaaataaacacatagaaataagtcccgatgaaatagttatcaagtttgtaggaaaggacaaagtttcacatgaatttgttgttcataagtctaatagactatataaaccgctattgaaactgacggatgattctagtcccgaagaatttctgttcaacaaactaagtgaacgaaaggtatacgaatgtatcaaacagtttggtattagaatcaaggatctccgaacgtatggagtcaattatacgtttttatataatttttggacaaatgtaaagtccatatctcctcttccgtcaccaaaaaagttaatagcattaactatcaaacaaactgctgaagttgtaggtcatactccatcaatttcaaaaagagcttatatggcaacgactattttagaaatggtaaaggataaaaattttttagatgtagtatctaaaactacgttcgatgaattcctatctatagtcgtagatcacgttaaatcatctacggatggatga

SEQ ID NO: 70 - DNA, 855 bp, Vaccinia virus
atggatctgtcacgaattaatacttggaagtctaagcagctgaaaagctttctctctagtaaagatacatttaaggcggatgtccatggacatagtgccttgtattatgcaatagctgataataacgtgcgtctagtatgtacgttgttgaacgctggagcattgaaaaatcttctagagaatgaatttccattacatcaggcagccacattggaagataccaaaatagtaaagattttgctattcagtggactggatgattcacaatttgatgacaagggaaacaccgcattgtattatgcggttgatagtggtaacatgcaaacggtaaaactgtttgttaagaaaaattggagactgatgttctatgggaaaactggatggaaaacttcattttatcatgccgtcatgcttaatgatgtaagtattgtttcctactttctttcagagataccatctacttttgatctggctattctccttagttgtattcacatcactataaaaaatggacacgtggatatgatgattctcttgctcgactatatgacgtcgacaaacaccaataattcccttctcttcattccggacattaaattggctatagataataaagacattgagatgttacaggctctgttcaaatacgacattaatatctattctgctaatctggaaaatgtactattggatgatgccgaaatagctaaaatgattatagaaaagcatgttgaatacaagtctgactcctatacaaaagatctcgatatagtcaagaataataaattggatgaaataattagcaaaaacaaggaactcagactcatgtacgtcaattgtgtaaagaaaaactaa

SEQ ID NO: 71 - DNA, 660 bp, Vaccinia virus
atggcgatgttttacgcacacgctctcggtgggtacgacgagaatcttcatgcctttcctggaatatcatcgactgttgccaatgatgtcaggaaatattctgttgtgtcagtttataataacaagtatgacattgtaaaagacaaatatatgtggtgttacagtcaggtgaacaagagatatattggagcactgctgcctatgtttgagtgcaatgaatatctacaaattggagatccgatccatgatcaagaaggaaatcaaatctctatcatcacatatcgccacaaaaactactatgctctaagcggaatcgggtacgagagtctagacttgtgtttggaaggagtagggattcatcatcacgtacttgaagcaggaaacgctgtatatggaaaagttcaacatgattattctactatcaaagagaaggccaaagaaatgagtacacttagtccaggacctataattgattaccacgtctggataggagattgtatctgtcaagttactgctgtggacgtacatggaaaggaaattatgaaaatgagattcaaaaagggtgcggtgcttccgatcccaaatctggtaaaagttaaacttggggagaatgatacagaaaatctttcttctactatatcggcggcaccatcgaggtaa

SEQ ID NO: 72 - DNA, 450 bp, Vaccinia virus
atgacggccaactttagtacccacgtcttttcaccacagcactgtggatgtgacagactgaccagtattgatgacgtcaaacaatgtttgactgaatatatttattggtcgtcctatgcataccgcaacaggcaatgcgctggacagttgtattccacactcctctcttttagagatgatgcggaattagtgttcatcgacattcgcgagctggtaaaacatatgccgtgggatgatgtcaaagattgtgcagaaatcatccgttgttatataccggatgagcaaaaaaccatcagagagatttcggccatcatcggactttgtgcatatgctgctacttactggggaggtgaagaccatcccactagtaacagtctgaacgcattgtttgtgatgcttgagatgctaaattacgtggattataacatcatattccggcgtatgaattga

SEQ ID NO: 73 - protein, 190 aa, Vaccinia virus
MSKIYIDERSDAEIVCAAIKNIGIEGATAAQLTRQLNMEKREVNKALYDLQRSAMVYSSDDIPPRWFMTTEADKPDADVMADAIIDDVSREKSMREDHKSFDDVIPAKKIIDWKDANPVTIINEYCQITKRDWSFRIESVGPSNSPTFYACVDIDGRVFDKADGKSKRDAKNNAAKLAVDKLLGYVIIRF

SEQ ID NO: 74 - protein, 90 aa, Vaccinia virus
MDMMLMIGNYFSGVLIAGIILLILSCIFAFIDFSKSTSPTRTWKVLSIMAFILGIIITVGMLIYSMWGKHCAPHRVSGVIHTNHSDISVN

SEQ ID NO: 75 - protein, 51 aa, Vaccinia virus
MNLQKLSLAIYLTATCSWCYETCIRKTALYHDIQLEHVEDNKDSVASLPYK

SEQ ID NO: 76 - protein, 147 aa, Vaccinia virus
MFNMNINSPVRFVKETNRAKSPTRQSPGAAGYDLYSAYDYTIPPGERQLIKTDISMSMPKICYGRIAPRSGLSLKGIDIGGGVIDEDYRGNIGVILINNGKCTFNVNTGDRIAQLIYQRIYYPELEEVQSLDSTNRGDQGFGSTGLR

SEQ ID NO: 77 - protein, 318 aa, Vaccinia virus
MTTVPVTDIQNDLITEFSEDNYPSNKNYEITLRQMSILTHVNNVVDREHNAAVVSSPEEISSQLNEDLFPDDDSPATIIERVQPHTTIIDDTPPPTFRRELLISEQRQQREKRFNITVSKNAEAIMESRSMITSMPTQTPSLGVVYDKDKRIQMLEDEVVNLRNQRSNTKSSDNLDNFTKILFGKTPYKSTEVNKRIAIVNYANLNGSPLSVEDLDVCSEDEIDRIYKTIKQYHESRKRKIIVTNVIIIVINIIEQALLKLGFEEIKGLSTDITSEIIDVEIGDDCDAVASKLGIGNSPVLNIVLFILKIFVKRIKII

SEQ ID NO: 78 - protein, 110 aa, Vaccinia virus
MDGTLFPGDDDLAIPATEFFSTKAAKKPEAKREAIVKADEDDNEETLKQRLTNLEKKITNVTTKFEQIEKCCKRNDEVLFRLENHAETLRAAMISLAKKIDVQTGRRPYE

SEQ ID NO: 79 - protein, 166 aa, Vaccinia virus
MGTAATIQTPTKLMNKENAEMILEKIVDHIVMYISDESSDSENNPEYIDFRNRYEDYRSLIIKSDHEFVKLCKNHAEKSSPETQQMIIKHIYEQYLIPVSEVLLKPIMSMGDIITYNGCKDNEWMLEQLSTLNFNNLRTWNSCSIGNVTRLFYTFFSYLMKDKLNI

SEQ ID NO: 80 - protein, 324 aa, Vaccinia virus
MAAVKTPVIVVPVIDRPPSETFPNVHEHINDQKFDDVKDNEVMPEKRNVVVVKDDPDHYKDYAFIQWTGGNIRNDDKYTHFFSGFCNTMCTEETKRNIARHLALWDSNFFTELENKKVEYVVIVENDNVIEDITFLRPVLKAMHDKKIDILQMREIITGNKVKTELVMDKNHAIFTYTGGYDVSLSAYIIRVTTALNIVDEIIKSGGLSSGFYFEIARIENEMKINRQILDNAAKYVEHDPRLVAEHRFENMKPNFWSRIGTAAAKRYPGVMYAFTTPLISFFGLFDINVIGLIVILFIMFMLIFNVKSKLLWFLTGTFVTAFI

SEQ ID NO: 81 - protein, 350 aa, Vaccinia virus
MNTRTDVTNDNIDKNPTKRGDRNIPGRNERFNDQNRFNNDIPKPKPRLQPNQPPKQDNKCREENGDFINIRLCAYEKEYCNDGYLSPAYYMLKQVDDEEMSCWSELSSLVRSRKAVGFPLLKAAKRISHGSMLYFEQFKNSKVVKLTPQVKCLNDTVIFQTVVILYSMYKRGIYSNEFCFNLVSIPRTNIVFSVNQLMFNICTDILVVLSICGNRLYRTNLPQSCYLNFIHGHETIARRGYEHSNYFFEWLIKNHISLLTKQTMDILKVKKKYATGAPVNRLLEPGTLVYVPKEDYYFIGISLTDVSISDNVRVLFSTDGIVLEIEDFNIKHLFMAGEMFVRSQSSTIIV

SEQ ID NO: 82 - protein, 241 aa, Vaccinia virus
MHVPASLQQSSSSCTEEENKHHMGIDVIIKVTKQDQTPTNDKICQSVTEITESESDPDPEVESEDDSTSVEDVDPPTTYYSIIGGGLRMNFGFTKCPQIKSISESADGNTVNARLSSVSPGQGKDSPAITHEEALAMIKDCEVSIDIRCSEEEKDSDIKTHPVLGSNISHKKVSYEDIIGSTIVDTKCVKNLEFSVRIGDMCKESSELEVKDGFKYVDGSASEGATDDTSLIDSTKLKACV

SEQ ID NO: 83 - protein, 108 aa, Vaccinia virus
MAEEFVQQRLANNKVTIFVKYTCPFCRNALDILNKFSFKRGAYEIVDIKEFKPENELRDYFEQITGGRTVPRIFFGKTSIGGYSDLLEIDNMDALGDILSSIGVLRTC

SEQ ID NO: 84 - protein, 101 aa, Vaccinia virus
MNSHFASAHTPFYINTKEGRYLVLKAVKVCDVRTVECEGSKASCVLKVDKPSSPACERRPSSPSRCERMNNPGKQVPFMRTDMLQNMFAANRDNVASRLLN

SEQ ID NO: 85 - protein, 72 aa, Vaccinia virus
MDTDVTNVEDIINEIDREKEEILKNVEIENNKNINKNHPNEYIREALVINTSSNSDSIDKEVIECICHDVGI

SEQ ID NO: 86 - protein, 53 aa, Vaccinia virus
MISNYEPLLLLVITCCVLLFNFTISSKTKIDIIFAVQTIVFIWFIFHFVHSAI

SEQ ID NO: 87 - protein, 269 aa, Vaccinia virus
MSKVIKKRVETSPRPTASSDSLQTCAGVIEYAKSISKSNAKCIEYVTLNASQYANCSSISIKLTDSLSSQMTSTFIMLEGETKLYKNKSKQDRSDGYFLKIKVTAASPMLYQLLEAVYGNIKHKERIPNSLHSLSVETITEKTFKDESIFINKLNGSMVEYVSTGESSILRSIEGELESLSKRERQLAKAIITPVVFYRSGTETKITFALKKLIIDREVVANVIGLSGDSERVSMTENVEEDLARNLGLVDIDDEYDEDSDKEKPIFNV

SEQ ID NO: 88 - protein, 133 aa, Vaccinia virus
MAEWHKIIEDISKNNKFEDAAIVDYKTTKNVLAAIPNRTFAKINPGEIIPLITNRNILKPLIGQKYCIVYTNSLMDENTYAMELLTGYAPVSPIVIARTHTALIFLMGKPTTSRRDVYRTCRDHATRVRATGN

SEQ ID NO: 89 - protein, 124 aa, Vaccinia virus
MKNVLIIFGKPYCSICENVSESVEELKSEYDILHVDILSFFLKDGDSSMLGDVKRGTLIGNFAAHLSNYIVSIFKYNPQTKQMAFVDINKSLDFTKTDKSLVNLEILKSEIEKATYGVWPPVTE

SEQ ID NO: 90 - protein, 314 aa, Vaccinia virus
MRALFYKDGKLFTDNNFLNPVLDDNPAYEVLQHVKIPTHLTDVVVYEQTWEEALTRLIFVGSDSKGRRQYFYGKMHVQNRNAKRDRIFVRVYNVMKRINCFINKNIKKSSTDSNYQLAVFMLMETMFFIRFGKMKYLKENETVGLLTLKNKHIEISPDEIVIKFVGKDKVSHEFVVHKSNRLYKPLLKLTDDSSPEEFLFNKLSERKVYECIKQFGIRIKDLRTYGVNYTFLYNFWTNVKSISPLPSPKKLIALTIKQTAEVVGHTPSISKRAYMATTILEMVKDKNFLDVVSKTTFDEFLSIVVDHVKSSTDG

SEQ ID NO: 91 - protein, 284 aa, Vaccinia virus
MDLSRINTWKSKQLKSFLSSKDTFKADVHGHSALYYAIADNNVRLVCTLLNAGALKNLLENEFPLHQAATLEDTKIVKILLFSGLDDSQFDDKGNTALYYAVDSGNMQTVKLFVKKNWRLMFYGKTGWKTSFYHAVMLNDVSIVSYFLSEIPSTFDLAILLSCIHITIKNGHVDMMILLLDYMTSTNTNNSLLFIPDIKLAIDNKDIEMLQALFKYDINIYSANLENVLLDDAEIAKMIIEKHVEYKSDSYTKDLDIVKNNKLDEIISKNKELRLMYVNCVKKN

SEQ ID NO: 92 - protein, 219 aa, Vaccinia virus
MAMFYAHALGGYDENLHAFPGISSTVANDVRKYSVVSVYNNKYDIVKDKYMWCYSQVNKRYIGALLPMFECNEYLQIGDPIHDQEGNQISIITYRHKNYYALSGIGYESLDLCLEGVGIHHHVLEAGNAVYGKVQHDYSTIKEKAKEMSTLSPGPIIDYHVWIGDCICQVTAVDVHGKEIMKMRFKKGAVLPIPNLVKVKLGENDTENLSSTISAAPSR

SEQ ID NO: 93 - protein, 149 aa, Vaccinia virus
MTANFSTHVFSPQHCGCDRLTSIDDVKQCLTEYIYWSSYAYRNRQCAGQLYSTLLSFRDDAELVFIDIRELVKHMPWDDVKDCAEIIRCYIPDEQKTIREISAIIGLCAYAATYWGGEDHPTSNSLNALFVMLEMLNYVDYNIIFRRMN

(19) United States
(12) Patent Application Publication
(10) Pub. No.: US 2006/0222658 A1
(43) Pub. Date: Oct. 5, 2006

(54) Vaccinia virus nucleic acids, polypeptides and immunogenic compositions

(76) Inventors: David Roth, San Diego, CA (US); Huaping He, San Diego, CA (US)

Correspondence Address: HELLER EHRMAN LLP, 275 MIDDLEFIELD ROAD, MENLO PARK, CA, 94025-3506, US

(21) Appl. No.: 11/291,305

(22) Filed: Nov. 30, 2005

Related U.S. Application Data
(60) Provisional application No. 60/632,548, filed on Dec. 1, 2004.

(51) Int. Cl. A61K 39/12; C07H 21/04; C12N 15/86; C12P 21/06; C07K 14/07

(52) U.S. Cl. 424/186.1; 536/23.72; 435/69.3; 435/320.1; 435/456; 530/350

(57) ABSTRACT

The present invention provides transcriptionally active vaccinia virus polynucleotides, recombinant vaccinia virus polypeptides, and immunogenic vaccinia virus antigens. Immunogenic compositions are also provided that may be useful as recombinant, subunit and DNA vaccines.

The present application claims the benefit under 35 U.S.C. 119(e) of the filing date of U.S. Application Ser. No. 60/632,548 filed on Dec. 1, 2004, incorporated by reference.

### STATEMENT REGARDING FEDERALLY SPONSORED RESEARCH AND DEVELOPMENT

The research leading to the present invention was supported, at least in part, by a grants from the National Institute of Allergy And Infectious Diseases. Accordingly, the Government may have certain rights in the invention.

### BACKGROUND OF THE INVENTION

1. Field of the Invention

The present invention relates to immunogenic vaccinia virus polypeptides, polynucleotides encoding immunogenic vaccinia virus polypeptides and immunogenic compositions comprising vaccinia virus polypeptides or polynucleotides.

2. Background

Traditional vaccine technology suffers from the problem that it often produces various degrees of reactogenicity in different hosts. Often, the only reliably immunogenic composition is a pathogenic microorganism. But the manufacture and administration of the pathogenic organism carries a risk of infection by the very pathogen the vaccine is designed to treat. Furthermore, recent well-publicized problems with influenza vaccine production highlight the difficulties in producing large quantities of conventional vaccines and the precarious state of worldwide vaccine supplies. In light of general health concerns and the growing threat of bioterrorism, there is a need to develop recombinant and subunit vaccines capable of inducing an appropriate immune response in the context of multiple and genetically diverse hosts. This approach requires the identification of a number of specific antigenic polypeptides. One of the most difficult tasks in developing a vaccine, or any recombinant subunit vaccine, is the identification of the antigens that can stimulate the most effective immune response against a particular pathogen, especially when the genome of the pathogen is large.

Because of its high case-fatality rates and transmissibility, Smallpox now represents one of the most serious bioterrorist threats. Over the centuries, naturally occurring Smallpox, with its case-fatality rate of 30 percent or more and its ability to spread in any climate and season, has been universally feared as one of the most devastating of all the infectious diseases. The use of Vaccinia virus as a vaccine enabled the global eradication of naturally occurring Smallpox. The last naturally occurring case of Smallpox occurred in Somalia in 1977. In May 1980, the World Health Assembly certified that the world was free of naturally occurring Smallpox. Routine vaccination against Smallpox in the United States ended in 1971, and except for some soldiers and laboratory workers, no one has been vaccinated since 1983. However, due to the present threat that Smallpox may be used as a weapon of terrorism, large-scale vaccination may once again be instituted.

Unfortunately, Smallpox vaccine has been manufactured using 25 year-old technology based on a Vaccinia virus used throughout the Smallpox eradication campaign. This technology comprises the harvesting of virus-containing material from live vaccinifers. Despite the proven safety record of this method of manufacture, various degrees of reactogenicity have been reported in vaccine recipients.

In the current age, where mass vaccination may be needed and immunosuppressive diseases are more prevalent than ever, a vaccine without the infective properties of a live virus is essential. Thus, there is a need for non-infectious Smallpox vaccines. In addition, there is a considerable need for additional diagnostic tests or tests to detect active vaccinia in the face of diseases such as smallpox.

### SUMMARY OF THE INVENTION

The present invention provides isolated polynucleotides encoding a vaccina virus polypeptide that is antigenic in a mammal, including SEQ ID NOS: 52-72 and fragments thereof, that encode antigenic polypeptides. The mammal can be, for example, a rabbit, human or mouse. The invention also provides isolated polynucleotides encoding highly immunogenic vaccina virus antigens including SEQ ID NOS: 52-72 and fragments thereof that encode immunogenic polypeptides. In some embodiments, highly immunogenic vaccina virus antigens react with polyclonal antibodies directed to vaccina virus bacteria from at least two different species. In another embodiment, highly immunogenic vaccina virus antigens are detected by ELISA, Western blotting or both using a polyclonal antibody that is directed to vaccina virus bacteria.

The present invention also provides TAP polynucleotides, e.g. polynucleotides produced by Transcriptionally-Activated PCR (TAP) technology as described in U.S. Pat. No. 6,280,977, which is expressly incorporated herein by reference. Such polynucleotides include a 5′TAP polynucleotide sequence, a vaccina virus polynucleotide sequence, and a 3′ TAP polynucleotide sequence. The vaccina virus polynucleotide sequence can, for example, comprise one of SEQ ID NOS: 52-72. In some embodiments, the 5′ TAP polynucleotide sequence comprises a promoter. In certain embodiments, the 5′ TAP polynucleotide sequence is selected from SEQ ID NOS: 2, 3, 6, and 8. In some embodiments the 3′TAP polynucleotide sequence comprises a terminator. In certain embodiments, the 3′TAP polynucleotide sequence is selected from SEQ ID NOS:4, 5, 7, and 9.

The present invention also provides primer pairs for amplifying a vaccina virus polynucleotide. These primer pairs include SEQ ID NOS: 10 and 11; 12 and 13; 14 and 15; 16 and 17; 18 and 19; 20 and 21; 22 and 23; 24 and 25; 26 and 27; 28 and 29; 30 and 31; 32 and 33; 34 and 35; 36 and 37; 38 and 39; 40 and 41; 42 and 43; 44 and 45; 46 and 47; 48 and 49, 50 and 51.

Isolated antigenic vaccina virus peptides and polypeptides are encompassed by the invention, including SEQ ID NOS: 73-93 and fragments thereof, that are antigenic peptide. Isolated vaccina virus peptides and polypeptides that are highly immunogenic, including SEQ ID NOS: 73-93 and fragments thereof, that are highly immunogenic, are also included in the invention. In one embodiment immunogenic peptides and polypeptides react with polyclonal antibodies that are directed to vaccina virus bacteria. In one aspect of this embodiment, the peptides and polypeptides react with polyclonal antibodies that are directed to vaccina virus bacteria from at least two different species. In another embodiment, highly immunogenic peptides and polypeptides are detected by ELISA, Western blotting or both using a polyclonal antibody that is directed to vaccina virus bacteria.

The present invention also includes recombinant vaccina virus peptides and polypeptides, wherein the amino terminus of the peptide or polypeptide comprises a HA tag or a His tag, and the carboxy terminus of the polypeptide is selected from the group consisting of: SEQ ID NOS: 73-93. Also included are recombinant vaccina virus peptides and polypeptides, wherein the carboxy terminus of the polypeptide comprises a HA tag or a His tag, and the amino terminus of the polypeptide is selected from the group consisting of: SEQ ID NOS: 73-93. The peptides and polypeptides of the invention may be expressed in an in vitro transcription-translation system, such as a T7 polymerase system.

Immunogenic compositions for inducing an immunological response in a mammalian host against vaccina virus, are also included in the invention. In one embodiment, the immunogenic compositions comprise nucleic acids that encodes and expresses in vivo in a mammalian host cell at least one immunogenic peptide or polypeptide, which may be any one of SEQ ID NOS: 73-93 or immunogenic fragments thereof. The nucleic acids can be, for example, plasmids or TAP fragments. The compositions can induce either a humoral or cell-mediated immune response. Furthermore, the immunogenic compositions can include additional components, such as adjuvants, as well as other applications such as serodiagnostics.

Immunogenic compositions for inducing an immunological response in a mammalian host against vaccinia of the invention can also comprise isolated vaccinia peptides and/or polypeptides, such as SEQ ID NOS: 73-93 or immunogenic fragments thereof. In one embodiment, the immunogenic peptides and polypeptides are expressed in an in vitro transcription and translation system. The immunogenic peptide and polypeptide compositions can induce either a humoral- or cell-mediated immune response. Furthermore, the immunogenic peptide and polypeptide compositions can include additional components, such as adjuvants, and include other applications such as diagnostics.

Similarly, detection of vaccinia, its constituent proteins, and/or its immunologically reactive products (e.g. antibodies) is clinically relevant for the diagnosis of vaccinia, and to track the efficacy of therapeutic treatments for vaccinia, especially as translated to serodiagnostic tests. The present application therefore provides antigens for detection of vaccinia for immune assays, including humoral immune assays. These antigens are applicable to detection of active vaccinia in the face of various diseases, such as smallpox, and rapidly mutating forms of vaccinia depending on genetic makeup, geographical location, and immunocompetency status.

As such, the present invention also provides diagnostic compositions, including one or more antibodies directed against the peptide epitopes identified herein. Also, the present invention provides diagnostic kits that include at least one or more of such antibodies.

The present invention also provides a method of generating an immune response in a mammalian host against vaccinia. The method includes: administering to a mammalian host an immunogenic composition comprising at least one nucleic acid selected from the group of SEQ ID NO: 52-72, fragments thereof or combinations thereof, wherein said nucleic acid encodes and expresses in vivo at least one immunogenic peptide or polypeptide, whereby said immune response against vaccinia is generated.

Also, the invention provides a method of generating an immune response in a mammalian host against vaccinia comprising: administering to said mammalian host an immunogenic composition comprising at least one nucleic acid encoding and expressing in vivo at least one immunogenic peptide or polypeptide selected from the group of SEQ ID NO: 73-93, fragments thereof or combinations thereof, whereby said immune response against vaccinia is generated.

In addition, the invention provides a method of generating an immune response in a mammalian host against vaccinia comprising administering to said mammalian host an immunogenic composition comprising at least one immunogenic peptide or polypeptide selected from SEQ ID NO: 73-93, fragment thereof or combinations thereof, whereby said immune response against vaccinia is generated.

Also, the invention provides kits. In one embodiment the invention provides a kit comprising at least one vaccinia immunogenic composition selected from a nucleic acid selected from the group consisting of SEQ ID NO: 52-72, fragments thereof or combinations thereof; or a peptide selected from the group consisting of SEQ ID NO: 73-93, fragments thereof or combinations thereof and an adjuvant. The kit may also include an expression system. In addition the kit may include at least 2, 5, 10, 15, 20 or more of said immunogenic compositions, including nucleic acids and/or peptides, combinations or fragments thereof. In addition, the kit may include controls, e.g. positive and/or negative controls.

### BRIEF DESCRIPTION OF THE DRAWINGS

FIG. 1 illustrates one method used to generate TAP Expression Fragments.

FIG. 2 displays a method of amplifying multiple genes using TAP technology, expressing said genes products, and purifying, and quantifying the resulting polypeptides.

FIG. 3 demonstrates how a plurality of polypeptides from a target organism can be assayed to determine each polypeptide's ability to elicit a humoral immune response.

FIG. 4 demonstrates how a plurality of polypeptides from a target organism can be assayed to determine each polypeptide's ability to elicit a cell mediated response.

FIG. 5 demonstrates that fluorescent proteins (goat IgG antibody) can be more effectively delivered into either NIH-3T3 cells (A&B) and human dendritic cells (C&D) with a protein delivery reagent (B&D) as opposed to without a protein delivery reagent (A&C).

FIGS. 6 and 6a show the results of scanning the vaccinia virus proteome for antigenic targets of humoral immunity by ELISA and Western blotting.

### DETAILED DESCRIPTION OF THE INVENTION

Before proceeding further with a description of the specific embodiments of the present invention, a number of terms will be defined and described in detail.

Unless specific definitions are provided, the nomenclature utilized in connection with, and the laboratory procedures, techniques and methods described herein are those known in the art to which they pertain. Standard chemical symbols and abbreviations are used interchangeably with the full names represented by such symbols. Thus, for example, the terms “carbon” and “C” are understood to have identical meaning. Standard techniques may be used for chemical syntheses, chemical analyses, pharmaceutical preparation, formulation, delivery, and treatment of patients. Standard techniques may be used for recombinant DNA methodology, oligonucleotide synthesis, tissue culture and the like. Reactions and purification techniques may be performed e.g., using kits according to manufacturer's specifications, as commonly accomplished in the art or as described herein. The foregoing techniques and procedures may be generally performed according to conventional methods well known in the art and as described in various general or more specific references that are cited and discussed throughout the present specification. All references cited herein are incorporated by reference in their entirety and are not admitted to be prior art. See e.g., Sambrook et al. Molecular Cloning: A Laboratory Manual (2d ed., Cold Spring Harbor Laboratory Press, Cold Spring Harbor, N.Y. (1989)), Harlow & Lane, Antibodies: A Laboratory Manual (Cold Spring Harbor Laboratory Press, Cold Spring Harbor, N.Y. (1988)), which are incorporated herein by reference in their entirety for any purpose.

The terms “polynucleotide” and “nucleic acid (molecule)” are used interchangeably to refer to polymeric forms of nucleotides of any length. The polynucleotides may contain deoxyribonucleotides, ribonucleotides and/or their analogs. Nucleotides may have any three-dimensional structure, and may perform any function, known or unknown. The term “polynucleotide” includes single-stranded, double-stranded and triple helical molecules. The following are non-limiting embodiments of polynucleotides: a gene, a gene fragment, exons, introns, mRNA, tRNA, rRNA, ribozymes, cDNA, recombinant polynucleotides, branched polynucleotides, plasmids, cosmids, viruses and other vectors, isolated DNA of any sequence, isolated RNA of any sequence, nucleic acid probes and primers. A nucleic acid molecule may also comprise modified nucleic acid molecules, such as methylated nucleic acid molecules and nucleic acid molecule analogs. Analogs of purines and pyrimidines are known in the art, and include, but are not limited to, aziridinylcytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudouracil, 5-pentyluracil and 2,6-diaminopurine. The use of uracil as a substitute for thymine in a deoxyribonucleic acid is also considered an analogous form of pyrimidine.

Sugar modifications (e.g., 2′-o-methyl, 2-fluoro and the like) and phosphate backbone modifications (e.g., morpholino, PNA′, thioates, dithioates and the like) can be incorporated singly, or in combination, into the nucleic acid molecules of the present invention. In one embodiment, for example, a nucleic acid of the invention may comprise a modified sugar and a modified phosphate backbone. In another embodiment, a nucleic acid of the invention may comprise modifications to sugar, base and phosphate backbone.

“Oligonucleotide” refers generally to polynucleotides of between 5 and about 100 nucleotides of single- or double-stranded nucleic acid, typically DNA. Oligonucleotides are also known as oligomers or oligos and may be isolated from genes, or synthesized (e.g., chemically or enzymatically) by methods known in the art.

A “primer” refers to an oligonucleotide, usually single-stranded, that provides a 3′-hydroxyl end for the initiation of enzyme-mediated nucleic acid synthesis.

“Peptide” generally refers to a short chain of amino acids linked by peptide bonds. Typically peptides comprise amino acid chains of about 2-100, more typically about 4-50, and most commonly about 6-20 amino acids. “Polypeptide” generally refers to individual straight or branched chain sequences of amino acids that are typically longer than peptides. “Polypeptides” usually comprise at least about 100 to about 1000 amino acids in length, more typically at least about 150 to about 600 amino acids, and frequently at least about 200 to about 500 amino acids. “Proteins” include single polypeptides as well as complexes of multiple polypeptide chains, which may be the same or different. Multiple chains in a protein may be characterized by secondary, tertiary and quaternary structure as well as the primary amino acid sequence structure; may be held together, for example, by disulfide bonds; and may include post-synthetic modifications such as, without limitation, glycosylation, phosphorylation, truncations or other processing. Antibodies such as IgG proteins, for example, are typically comprised of four polypeptide chains (i.e., two heavy and two light chains) that are held together by disulfide bonds. Furthermore, proteins may include additional components such as associated metals (e.g., iron, copper and sulfur), or other moieties. The definitions of peptides, polypeptides and proteins include, without limitation, biologically active and inactive forms; denatured and native forms; as well as variant, modified, truncated, hybrid, and chimeric forms thereof. The peptides, polypeptides and proteins of the present invention may be derived from any source or by any method, including, but not limited to extraction from naturally occurring tissues or other materials; recombinant production in host organisms such as bacteria, fungi, plant, insect or animal cells; and chemical synthesis using methods that will be well known to the skilled artisan.

“Polyclonal antibodies” or “antisera” are heterogeneous populations of antibody molecules derived from the sera of animals immunized with an antigen, or an antigenic functional derivative thereof. For the production of polyclonal antibodies, host animals such as rabbits, mice and goats, may be immunized by injection with an antigen or hapten-carrier conjugate optionally supplemented with adjuvants. Polyclonal antibodies may also be derived from the sera of humans or non-human animals exposed to a pathogen or vaccinated against a pathogen using a commercially available or experimental vaccine. An antiserum against vaccinia virus, for example, may be obtained from a human patient vaccinated with a vaccinia virus vaccine or from an animal, such as a mouse, rabbit, goat or sheep immunized with a vaccinia virus preparation

“Monoclonal antibodies,” which are homogeneous populations of antibodies to a particular antigen, may be obtained by any technique that provides for the production of antibody molecules by continuous cell lines in culture. These include, but are not limited to the hybridoma technique of Köhler and Milstein, Nature, 256:495-7 (1975); and U.S. Pat. No. 4,376,110, the human B-cell hybridoma technique (Kosbor, et al., Immunology Today, 4:72 (1983); Cote, et al., Proc. Natl. Acad. Sci. USA, 80:2026-30 (1983), and the EBV-hybridoma technique (Cole, et al., in Monoclonal Antibodies And Cancer Therapy, Alan R. Liss, Inc., New York, pp. 77-96 (1985)). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this a presently preferred method of production.

In addition, techniques developed for the production of “chimeric antibodies” (Morrison, et al., Proc. Natl. Acad. Sci., 81:6851-6855 (1984); Takeda, et al., Nature, 314:452-54 (1985)) by splicing the genes from a mouse antibody molecule of appropriate antigen specificity together with genes from a human antibody molecule of appropriate biological activity can be used. A chimeric antibody is a molecule in which different portions are derived from different sources, such as those having a variable region derived from a murine mAb and a human immunoglobulin constant region. Humanized antibodies can also be generated in which certain parts (e.g., framework regions) of a non-human antibody are altered to make the antibody more like a human antibody, while retaining antigen binding features of the parent molecule.

Alternatively, techniques described for the production of single chain antibodies (U.S. Pat. No. 4,946,778; Bird, Science 242:423-26 (1988); Huston, et al., Proc. Natl. Acad. Sci. USA, 85:5879-83 (1988); and Ward, et al., Nature, 334:544-46 (1989)) can be adapted to produce single chain antibodies. Single chain antibodies are typically formed by linking the heavy and light chain fragments of the Fᵥ region via an amino acid bridge, resulting in a single chain polypeptide.

Antibody fragments that recognize specific epitopes may be generated by known techniques. For example, such fragments include but are not limited to: the Fab fragments that can be produced by papain digestion of the antibody molecule, the F(ab′)₂ fragments that can be produced by pepsin digestion of the antibody molecule and the Fab′ fragments that can be generated by reducing the disulfide bridges of the F(ab′)₂ fragments. Alternatively, Fab expression libraries may be constructed (Huse, et al., Science, 246:1275-81 (1989)) to allow rapid and easy identification of monoclonal Fab fragments with the desired specificity.

The term “hapten” as used herein, refers to a small proteinaceous or non-protein antigenic determinant which is capable of being recognized by an antibody. Typically, haptens do not elicit antibody formation in an animal unless part of a larger species. For example, small peptide haptens are frequently coupled to a carrier protein such as keyhole limpet hemocyanin in order to generate an anti-hapten antibody response. “Antigens” are macromolecules capable of generating an antibody response in an animal and being recognized by the resulting antibody. Both antigens and haptens comprise at least one antigenic determinant or “epitope,” which is the region of the antigen or hapten which binds to the antibody. Typically, the epitope on a hapten is the entire molecule.

By the terms “specifically binding” and “specific binding” as used herein is meant that an antibody or other molecule, binds to a target such as an antigen, with greater affinity than it binds to other molecules under the specified conditions of the present invention. Antibodies or antibody fragments, as known in the art, are polypeptide molecules that contain regions that can bind other molecules, such as antigens. In various embodiments of the invention, “specifically binding” may mean that an antibody or other specificity molecule, binds to a target molecule with at least about a 10⁶-fold greater affinity, preferably at least about a 10⁷-fold greater affinity, more preferably at least about a 10⁸-fold greater affinity, and most preferably at least about a 10⁹-fold greater affinity than it binds molecules unrelated to the target molecule. Typically, specific binding refers to affinities in the range of about 10⁶-fold to about 10⁹-fold greater than non-specific binding. In some embodiments, specific binding may be characterized by affinities greater than 10⁹-fold over non-specific binding. Whenever a range appears herein, as in “1-10 or one to ten, the range refers without limitation to each integer or unit of measure in the given range. Thus, by 1-10 it is meant each of 1, 2, 3, 4, 5, 6, 7, 8, 9, 10 and any subunit in between.

“Immunogenic compositions” of the present invention are preparations that, when administered to a human or non-human animal, elicit a humoral and/or cellular immune response. “Vaccine,” as used herein, refers to immunogenic compositions that are administered to a human or non-human patient for the prevention, amelioration or treatment diseases, typically infectious diseases. “Traditional vaccines” or “whole vaccines” typically may be live, attenuated or killed microorganisms, such as bacteria or viruses. Vaccines also encompass preparations that elicit or stimulate an immune response that may be useful in the prevention, amelioration or treatment of non-infectious diseases. For example, a cancer cell vaccine may be administered to stimulate or supplement a patient's immune response to neoplastic disease. “Subunit vaccines” may be prepared from purified or partially purified proteins or other antigens from a microorganism, cancer cell or other vaccine target. The term “recombinant vaccine” refers to any vaccine that is prepared using recombinant DNA technology and includes certain subunit vaccines (for example, where subunits are cloned and expressed in vitro prior to administration) and “polynucleotide vaccines” such as DNA vaccines, that may encode immunogenic polypeptides. Vaccines typically contain at least one immunogenic component (e.g. a cell, virus, polypeptide, polynucleotide, and the like) but may also include additional agents such as adjuvants, which may enhance or stimulate the patient's immune response to the immunogenic component. In certain embodiments, vaccines or components of vaccines may be conjugated e.g. to a polysaccharide or other molecule, to improve stability or immunogenicity of one or more vaccine components.

As used herein, the term “promoter” refers to a DNA sequence having a regulatory function, which is recognized (directly or indirectly) and bound by a DNA-dependent RNA polymerase during the initiation of transcription. Promoters are typically adjacent to the coding sequence of a gene and extend upstream from the transcription initiation site. The promoter regions may contain several short (<10 base pair) sequence elements that bind transcription factors, generally located within the first 100-200 nucleotides upstream of the transcription initiation site. Sequence elements that regulate transcription from greater distances are generally referred to as “enhancers” and may be located several hundred or thousand nucleotides away from the gene they regulate. Promoters and enhancers may be cell- and tissue-specific; they may be developmentally-programmed; they may be constitutive or inducible e.g., by hormones, cytokines, antibiotics, physiological and metabolic states. For example, the human metallothionein (MT) promoter is upregulated by heavy metal ions and glucocorticoids. Inducible promoters and other elements may be operatively positioned to allow the inducible control or activation of expression of the desired TAP fragment. Examples of such inducible promoters and other regulatory elements include, but are not limited to, tetracycline, metallothionine, ecdysone, and other steroid-responsive promoters, rapamycin responsive promoters, and the like (see e.g., No, et al., Proc. Natl. Acad. Sci. USA, 93:3346-51 (1996); Furth, et al., Proc. Natl. Acad. Sci. USA, 91:9302-6 (1994)). Certain promoters are operative in prokaryotic cells, while different promoter sequences are required for transcription in eukaryotic cells. Additional control elements that can be used include promoters requiring specific transcription factors, such as viral promoters which may require virally encoded factors. Promoters can be selected for incorporation into TAP fragments based on the intended use of the polynucleotide, as one skilled in the art will readily appreciate. For example, if the polynucleotide encodes a polypeptide with potential utility in human cells, then a promoter capable of promoting transcription in mammalian cells can be selected. Typical mammalian promoters include muscle creatine kinase promoter, actin promoter, elongation factor promoter as well as those found in mammalian viruses such as CMV, SV40, RSV, MMV, HIV, and the like. In certain embodiments, it may be advantageous to incorporate a promoter from a plant or a plant pathogen (e.g., cauliflower mosaic virus promoter), a promoter from a fungus such as yeast (e.g., Gal 4 promoter), a promoter from a bacteria or bacterial virus, such as bacteriophage lambda, T3, T7, SP6, and the like.

The term “terminator” refers to DNA sequences, typically located at the end of a coding region, that cause RNA polymerase to terminate transcription. As used herein, the term “terminator” also encompasses terminal polynucleotide sequences that direct the processing of RNA transcripts prior to translation, such as, for example, polyadenylation signals. Any type of terminator can be used for the methods and compositions of the invention. For example, TAP terminator sequences can be derived from a prokaryote, eukaryote, or a virus, including, but not limited to animal, plant, fungal, insect, bacterial and viral sources. In one embodiment, artificial mammalian transcriptional terminator elements are used. A nonexclusive list of terminator sequences that may be used in the present invention include the SV40 transcription terminator, bovine growth hormone (BGH) terminator, synthetic terminators, rabbit β-globin terminator, and the like. Terminators can also be a consecutive stretch of adenine nucleotides at the 3′end of a TAP fragment.

By “pharmaceutically acceptable” or “pharmacologically acceptable” is meant a material which is not biologically or otherwise undesirable, i.e., the material may be administered to an individual in a formulation or composition without causing any undesirable biological effects or interacting in a deleterious manner with any of the components of the composition in which it is contained.

By “serodiagnostic test” or grammatical equivalents herein is meant diagnostic tests to detect vaccinia by serum of infected organisms, animals or patients.

By “diagnostic test” or grammatical equivalents herein is meant an assay or test to detect vaccinia by any scientific technique from infected organisms, animals or patients.

Overview

The present invention generally relates to vaccinia virus polypeptide libraries, methods of determining the immunogenic effect of vaccinia virus polypeptides, methods of developing vaccines against vaccinia virus, immunogenic and pharmaceutical compositions. The invention also provides immunogenic vaccinia virus polypeptides and mixtures of polypeptides, polynucleotides encoding immunogenic vaccinia virus polypeptides and immunogenic compositions comprising vaccinia virus polypeptides or polynucleotides.

Polypeptide Libraries

According to a method of the present invention, a library or array of vaccinia polypeptides, oligonucleotides, or polynucleotides is generated. The immunogenicity of individual polypeptides in the library or array is determined by immunological screening where suitable, highly immunogenic vaccinia polypeptides are selected for vaccine development. Conveniently, individual polypeptides in the library may be arranged in an array to facilitate screening in a rapid and high throughput manner.

The term “array” includes any arrangement wherein a plurality of different molecules, compounds or other species are contained, held, presented, positioned, situated, or supported. Arrays can be arranged on microtiter plates, such as 48-well, 96-well, 144-well, 192-well, 240-well, 288-well, 336-well, 384-well, 432-well, 480-well, 576-well, 672-well, 768-well, 864-well, 960-well, 1056-well, 1152-well, 1248-well, 1344-well, 1440-well, or 1536-well plates, tubes, slides, chips, flasks, or any other suitable laboratory apparatus. In one embodiment, molecules arranged in an array are peptides, polypeptides or proteins. In another embodiment, the molecules are oligonucleotides or polynucleotides. In one aspect of the invention, polypeptides or polynucleotides in solution are arranged in 96 well plate arrays. In another embodiment, polypeptides or polynucleotides are immobilized on a solid support in an array format. Furthermore, an array can be sub-divided into a plurality of sub-arrays, as for example, where multiple 96-well plates (each an individual sub-array) are required to hold all of the samples of a single, large array.

The term “library” is likewise to be construed broadly, and includes any non-naturally occurring collection of molecules, whether arranged or not. A library therefore encompasses an array but the two terms are not necessarily synonymous.

TAP Technology

Libraries of vaccinia virus polypeptides may be prepared by any method known in the art. Conveniently, Transcriptionally Active Polymerase Chain Reaction (“TAP”) can be used to amplify DNA in preparation for producing vaccinia polypeptide libraries. With TAP technology, a particular polynucleotide of interest can be made transcriptionally active and ready for expression in less than one day. “TAP fragments” are transcriptionally active coding sequences prepared using TAP technology, and the two terms can be used interchangeably. TAP fragments encompass polynucleotides that can be readily expressed, for example, by transfection into animal cells or tissues by any nucleic acid transfection technique, without the need for subcloning into expression vectors or purification of plasmid DNA from bacteria. TAP fragments can be synthesized by amplification (e.g., polymerase chain reaction or PCR) of any polynucleotide of interest using nested oligonucleotide primers. Two polynucleotide sequences are typically incorporated into TAP fragments, one of which comprises an active transcriptional promoter and the other comprises a transcriptional terminator.

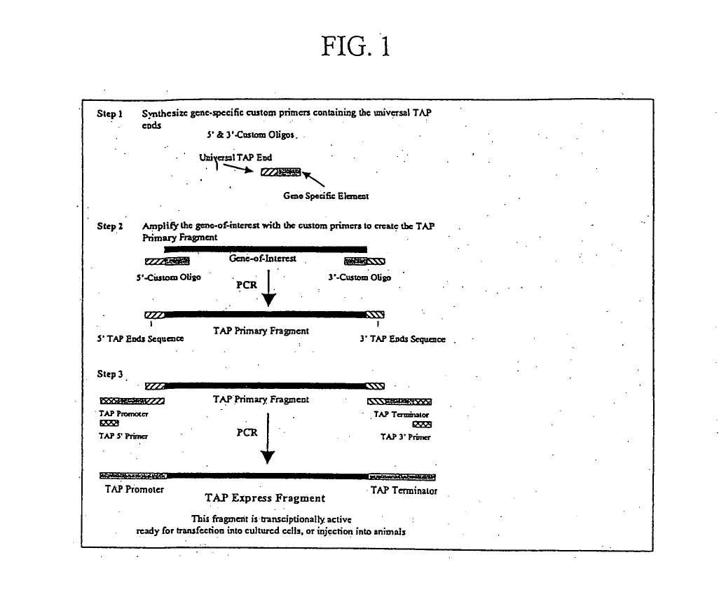

TAP fragments and methods of making the same are described in detail in U.S. Pat. No. 6,280,977, entitled “Method for Generating Transcriptionally Active DNA Fragments” which is hereby incorporated by reference in its entirety. In one embodiment, methods for creating TAP fragments include the steps of: i) designing oligonucleotide primers; ii) amplifying TAP primary fragments; and iii) amplifying TAP expression fragments. FIG. 1 illustrates one method for generating TAP fragments.

TAP fragments can be prepared using custom oligonucleotide primers designed to amplify a target polynucleotide sequence of interest from the vaccinia virus genome. Primers complementary to the 5′ and 3′ends of the polynucleotide of interest can be designed and synthesized using methods well known in the art, and can include any suitable number of nucleotides to permit amplification of the coding region. Typically, the polynucleotide sequence of interest is an open reading frame (ORF) that consists of an uninterrupted stretch of triplet amino acid codons, without stop codons. In certain embodiments, the polynucleotide is a vaccinia virus polypeptide-encoding sequence.

In one embodiment of the invention, 5′-custom oligonucleotide primers of about 41, 42, 43, 44, 45 or 46 nucleotides are designed and synthesized; about 36 nucleotides of which comprise the 5′-TAP end universal sequence 5′-GAAGGAGATATACCATGCATCATCATCATCATCAT-3′ (SEQ ID NO: 8) and about 15 to 20 nucleotides are complementary to the vaccinia virus sequence. Accordingly, the target-specific sequence can be, for example, about 15, 16, 17, 18, 19, or 20 nucleotides in length. The 5′ oligonucleotide may also incorporate a Kozak consensus sequence (A/GCCAUGG) near an ATG start codon (initiator methionine) for more efficient translation of mRNA. In one embodiment, an ATG start codon is included in the target-specific primer sequence. In another embodiment, an ATG start codon is incorporated into the custom 5′-oligonucleotide when the target sequence encoding a polypeptide of interest lacks an initiation methionine codon at its 5′end.

In one embodiment of the invention, 3′-custom oligonucleotide primers comprise about 35, 36, 37, 38, 39, 40, 41, 42, 43, 44, or 45 nucleotides; of these, about 20 nucleotides comprise the 3′-TAP end universal sequence 5′-TGATGATGAGAACCCCCCCC-3′ (SEQ ID NO: 9) and about 20 nucleotides are complementary to the target vaccinia virus sequence. In one aspect, a stop codon sequence, can be added to the end of the target vaccinia virus sequence to achieve proper translational termination by incorporating a TCA, TTA, or CTA into the 3′-custom oligonucleotide.

Bioinformatics Analysis of Vaccinia Virus Polynucleotides

In one embodiment of the invention, a bioinformatics approach is used to identify, prioritize and select vaccinia virus genes, coding sequences, open reading frames and other sequences of interest for TAP amplification and to design custom 5′ and 3′oligonucleotide primers. According to this approach, a database of vaccinia virus genomic information is compiled from available nucleic acid and amino acid sequence information, including the polynucleotide, gene, locus, polypeptide, and protein names, locations and sizes. In certain embodiments, the location of known coding sequences is included in the database. The sequence information may also be analyzed for unidentified open reading frames and putative coding sequences. Any method can be used to identify ORFs and coding sequences including free or commercially available sequence analysis software. For example, the GLIMMER program may be used to predict putative coding regions or genes in prokaryotic nucleotide sequences. See e.g., Salzberg et al., Nucleic Acids Res. 26: 544-548 (1998); Delcher, et al., Nucleic Acids Res. 27:4636-4641(1999).

In certain aspects of the invention, the genome database includes the entire genomic DNA sequence of vaccinia virus. In one embodiment, the sequence information is obtained from information that is in the public domain. In other embodiments, some or all of the sequence information can be obtained by nucleotide and/or amino acid sequencing.

As previously described in U.S. Ser. No. 10/159,428, which is hereby incorporated by reference in its entirety, the methods of the present invention, particularly TAP technology, enable the skilled artisan to prepare a library representing all or substantially all of the polypeptides expressed in an organism or cell type. In certain embodiments of the present invention, however, it may be preferable to prepare a library of polypeptides with selected properties. Thus, one aspect of the present invention utilizes a set of ranking criteria to identify polypeptides predicted to have properties desirable e.g., for vaccine development. Polypeptide ranking criteria, which may be identified using bioinformatics tools, include but are not limited to, the presence of membrane domains, length of ORF, secreted proteins signatures, signal sequences, hydrophobicity, B-cell and T-cell epitopes, homology to human proteins, protein and gene expression levels. The ranking criteria may be assigned a numerical score based on relative importance. Coding regions or putative coding regions identified in the database of vaccinia virus sequences are then scored using the numerical ranking criteria and the sum of the scores for each sequence is used to establish a rank order. According to this aspect of the invention, primers are designed to amplify vaccinia virus polynucleotides in rank order. A library may be constructed, for example, from the top 5%, 10%, 20%, 30%, 40% or 50% by rank of vaccinia virus polynucleotides.

Amplification of Vaccinia Virus Polynucleotides

Using the custom 5′ and 3′ oligonucleotide primers, TAP primary fragment may be amplified by methods well known in the art. The term “TAP primary fragment” refers to an amplified vaccinia virus polynucleotide, and in one embodiment relates to a polynucleotide sequence that has been amplified but is not transcriptionally active. Generation of TAP primary fragments involves performing PCR, which generates a polynucleotide fragment that contains the vaccinia virus polynucleotide sequence with 5′- and 3′-TAP universal end sequences and may contain other sequences incorporated into the custom 5′ and 3′ oligonucleotide primers. The 5′- and 3′-TAP universal end sequences are particularly useful for incorporating one or more nucleotide sequences into TAP primary fragment that confer transcriptional activity. In one embodiment, these sequences can include TAP Express™ promoter and terminator fragments (e.g., SEQ ID NOS:2-7). The skilled artisan will be familiar with methods for amplifying polynucleotides, (e.g. by using PCR) and can adjust the above methods in order to optimize the amplification reaction.

The generation of TAP fragments involves incorporating at least one polynucleotide sequence that confers transcriptional activity into the TAP primary fragment. Typically, the at least one polynucleotide sequence is incorporated by performing a second PCR reaction. Examples of polynucleotide sequences that confer transcriptional activity are promoter sequences (e.g., prokaryotic Pribnow boxes and eukaryotic TATA box sequences) binding sites for transcription factors, and enhancers. In one embodiment, one promoter and one terminator sequence are added to the TAP fragment. These promoter and terminator sequences can be obtained in numerous ways. For example, one can use restriction enzyme digestion of commercially available plasmids and cDNA molecules, or one can synthesize these sequences with an automated DNA synthesizer by methods well known in the art.

The end product of the second PCR reaction, is referred to as a “TAP expression fragment,” which is a transcriptionally active polynucleotide, and which is generally a transcriptionally active coding sequence. In certain embodiments, the TAP expression fragments are used directly for in vivo or in vitro (e.g. cell-free) expression. In other embodiments, TAP expression fragments are transfected into cultured cells or injected into animals.

Generating TAP fragments is a rapid and efficient way of making a large number of polynucleotide sequences transcriptionally active. Accordingly, a plurality of different genes from vaccinia virus can be made transcriptionally active using TAP technology. Thus, a library representing all, substantially all, or a selected subset of the coding sequences in the vaccinia virus genome can be constructed using TAP technology.

TAP Tags and Linker Molecules

As described above, TAP technology provides powerful methods for amplifying and expressing vaccinia virus polynucleotides. Coding sequences can be rendered transcriptionally active by the PCR-mediated addition of promoters sequences, enhancers, terminators and other regulatory sequences.

In addition, vaccinia virus polynucleotides can be amplified with additional coding or non-coding sequences that can facilitate rapid screening, characterization, purification and study of the polypeptides that they encode. These additional sequences include for example, reporter genes, affinity tags, antibody tags, PNA binding sites, secretory signals, and the like.

According to the present invention, vaccinia virus polynucleotides can be synthesized with an epitope tag. An “epitope tag” is a short stretch of polynucleotide sequence encoding an epitope. In one embodiment, this epitope is recognized by a well-characterized antibody. By incorporating an epitope tag into TAP fragments, the vaccinia virus polynucleotide of interested can be fused in-frame to an epitope-encoding sequence. Expression of an epitope-tagged TAP fragment produces a fusion protein comprising a tagged vaccinia virus polypeptide. Suitable epitope tags will be well known to those skilled in the art, including the hemagglutinin (HA) epitope tag and the Flag epitope tag. The HA epitope tag is well characterized and highly immunoreactive. Upon transfection of an HA-tagged TAP fragment into cells, the resulting HA-tagged polypeptides can be identified with commercially available anti-HA antibodies. Epitope tagging of TAP fragments is useful for rapidly and conveniently detecting expression of TAP fragments. Epitope tagging of TAP fragments can also help determine the intracellular distribution of vaccinia virus polypeptides and help characterize and purify the vaccinia virus polypeptide. Furthermore, HA-tagged expression products can be quickly captured and/or purified using antibodies specific for the HA epitope. Antibodies directed against the HA epitope can be used in the full range of immunological techniques for detection and analysis of tagged polypeptides including but not limited to Western blotting, ELISAs, radioimmune assays, immunoprecipitation, immunocytochemistry and immunofluorescence, fluorescence assisted cell sorting (FACS) and immunoaffinity purification of the desired fusion polypeptides.

The present invention also provides vaccinia virus polypeptides fused to affinity tags. For example, a polynucleotide sequence encoding a histidine tag can be incorporated into the TAP fragment to enable the expressed gene product to be conveniently purified. His tags consisting of six consecutive histidine residues (6×His) are a powerful tool for recombinant polypeptide purification. The 6×His tag interacts with metals, such as nickel. Thus, polypeptides fused to a 6×His tag can be purified by metal affinity chromatography, for example, using a nickel nitrilotriacetic (Ni-NTA) resin. The 6×His tag is much smaller than most other affinity tags and is uncharged at physiological pH. It rarely alters or contributes to polypeptide immunogenicity, rarely interferes with polypeptide structure or function, does not interfere with secretion, does not require removal by protease cleavage, and is compatible with denaturing buffer systems. Accordingly, this tag is a powerful adjunct to expression and purification of recombinant proteins.

In one aspect of this embodiment, TAP primers can be designed to include the nucleotide sequence encoding the 6×His epitope tag: to add the 6×His epitope to the 5′ end of a vaccinia virus polynucleotide, a sequence encoding histidine residues can be included along with the promoter-containing primer; to add the 6×His epitope to the 3′end, the His sequence can be included in the terminator-containing primer.

Commercially available nickel affinity resins can be used to purify 6×His tagged polypeptides. For example, the well-established QIAexpress Protein Expression and Purification Systems are based on the remarkable selectivity and affinity of patented nickel-nitrilotriacetic acid (Ni-NTA) metal-affinity chromatography matrices for polypeptides tagged with 6 consecutive histidine residues (6×His tag) available from QIAGEN (Seattle, Wash.). The QIAexpress System is based on the remarkable selectivity of Ni-NTA (nickel-nitrilotriacetic acid) for polypeptides with an affinity tag of six consecutive histidine residues—the 6×His tag. This technology allows purification, detection, and assay of almost any 6×His-tagged polypeptide from any expression system. Polypeptides with a 6×His tag can be purified through nickel nitrilotriacetic (Ni-NTA) resin.

The HA and the 6×His epitope embodiments are not to be construed as limiting, and are provided for illustrative purposes only. Those skilled in the art will appreciate that any type of tag can be attached to the expressed products such as for example, a 7×, 8×, 9×, or 10× histidine tag, GST tag, fluorescent protein tag, and the like.

In addition to providing a convenient means for detection and purification of vaccinia virus polypeptides, various tags can provide a “linker” through which the polypeptides of the invention can be immobilized on a solid support. The term “linker molecule,” as used herein, encompasses any molecule that is capable of immobilizing the polypeptides to a solid support.

TAP Fragment with Secretory Signal

For many gene therapy and DNA vaccine applications it may be beneficial for the gene product to be secreted from the transfected cells. Thus, one embodiment of the invention provides a version of the TAP system designed to express vaccinia virus polypeptides containing a fused a secretory signal. A commonly used signal peptide is the first 23 amino acids from human tissue plasminogen activator (tPA) with the coding sequence as follows: ATG GAT GCA ATG AAG AGA GGG CTC TGC TGT GTG CTG CTG CTG TGT GGA GCA GTC TTC GTT TCG CCC AGC. (SEQ ID NO: 1). This sequence can be built into the TAP promoter fragment to create a new TAP fragment in a fashion similar to the construction of the tagged polypeptides described above.

Incorporating TAP Fragments into a Plasmid Vector

Once the function or immunogenicity of a vaccinia virus polypeptide is identified, it may be of desirable to clone the TAP fragment into a plasmid or other vector to facilitate further gene characterization and manipulation.

Standard cloning techniques can involve the use of restriction enzymes to digest the plasmid and the gene fragment to be inserted. Annealing and ligation of the compatible ends can lead to insertion of the gene into the vector. An alternative method of restriction ends-directed cloning is to prepare a linearized plasmid with T overhangs on the 3′ends of the double-stranded DNA to accommodate DNA fragments amplified with the aid of specific polymerases through PCR. This method is sometimes called “T/A cloning”. Other methods of cloning TAP fragments will be well known to those of skill in the art.

In certain embodiments, the TAP Cloning systems, methods, and kits can further simplify the cloning process by taking advantage of the universal 5′ and 3′ sequences that are present on the TAP Express fragment after the first or second PCR step. These regions overlap with the end sequences of our linearized TAP Express Cloning Vector. When the TAP fragment and the linearized plasmid are mixed together and directly electroporated into TAP Express Electro-Comp cells, endogenous bacterial recombinase activity recombines the two DNA fragments resulting in a plasmid with the inserted TAP Express fragment. This process can replace conventional cloning with two simple PCR steps. In some embodiments it does not require cutting, pasting and ligating DNA fragments. In addition, this process can be highly suited for fast and convenient cloning of TAP PCR fragments without having to resort to restriction enzymes, DNA ligase, Topo-isomerase or other DNA modifying enzymes. “TAP” systems, vectors and cells are readily available from Gene Therapy Systems, San Diego, Calif.

GeneGrip PNA compatible TAP system can also be used to couple polypeptides onto DNA through PNA Dependent Gene Chemistry, thereby avoiding many of the limitations of previously described methodologies. GeneGrip is available through Gene Therapy Systems, San Diego, Calif. This approach takes advantage of the property of peptide nucleic acids (PNA) to hybridize with duplex DNA in a sequence specific and very high affinity manner. PNA binding sites can be used for attaching a series of peptides onto DNA in order to target the transfected plasmid and improve transgene expression, for example. This can facilitate a rational approach to improve the efficiency and efficacy of gene delivery by adding elements intended to increase nuclear uptake, facilitate endosomal escape, or target gene delivery to the cell surface or to intracellular receptors.

Incorporating a GeneGrip site into TAP enables peptide nucleic acids (PNAs) to be hybridized to the TAP gene. Ligands can then be attached to the PNA in order to improve the bioavailability and DNA vaccine potency of the gene.

System for Performing TAP Method

In another embodiment of the invention, a system can be used to perform every step involved in generating TAP fragments from a vaccinia virus. Additionally, each individual step is capable of being controlled by a system. For example, a system can design customized PCR primers, obtain said primers, perform PCR reactions utilizing TAP technology, attach promoters and terminators, and attach sequences that encode linker molecules to the primary or expression fragment. The system can be either automated or nonautomated. In one embodiment of the invention, the system comprises a computer program linked to robotic technologies for rapid and high throughput gene amplification of the genome.

Expression of the TAP Fragment

TAP fragments can be used directly as templates in various expression systems in order to obtain the corresponding polypeptide for each coding sequence in the vaccinia virus genome. The invention provides simple, efficient methods for generating TAP fragments from vaccinia virus that can be readily transfected into animal cells or tissues by any nucleic acid transfection technique. The methods of the invention can avoid the need for subcloning into expression vectors and for purification of plasmid DNA from bacteria. As skilled artisans can appreciate, TAP fragments can be rapidly expressed using in vivo or in vitro (e.g. cell-free) expression systems. For example, the amplified TAP fragments can be directly transfected into a eukaryotic or prokaryotic cell for expression. Examples of eukaryotic cells that can be used for expression include mammalian, insect (e.g. Baculovirus expression systems), yeast (e.g. Picchia pastoris), and the like. An example of a prokaryotic cell expression system includes E. coli.

Alternatively, expression can be accomplished in cell-free systems, for example, a T7 transcription-translation systems. Cell-free translation systems can include extracts from rabbit reticulocytes, wheat germ and Escherichia coli. These systems can be prepared as crude extracts containing the macromolecular components (70S or 80S ribosomes, tRNAs, aminoacyl-tRNA synthetases, initiation, elongation and termination factors, etc.) required for translation of exogenous RNA. To promote efficient translation, each extract can be supplemented with amino acids, energy sources (ATP, GTP), energy regenerating systems (creatine phosphate and creatine phosphokinase for eukaryotic systems, and phosphoenol pyruvate and pyruvate kinase for the E. coli lysate), and other co-factors (Mg²⁺, K⁺, etc.).

The use of TAP technology allows skilled artisans to rapidly express polypeptides from a plurality of polynucleotides. After a particular vaccinia virus polynucleotide of interest is rendered transcriptionally active, other vaccinia virus polynucleotides can also be made to be transcriptionally active according to the methods of the invention. Accordingly, in one embodiment of the invention, a plurality of polynucleotides from vaccinia virus are amplified and expressed in order to generate a library of vaccinia virus polypeptides.

Other embodiments of the invention relate to expressing the product of a vaccinia virus polynucleotide that encodes an epitope tag, affinity tag or other tags, and which may function as linkers. A polynucleotide sequence encoding a linker molecule can be incorporated into a TAP primary fragment or a TAP expression fragment. Accordingly, the linker molecule can be expressed as a fusion to the vaccinia virus polypeptide.

The generation of vaccinia virus polypeptide libraries according to the methods of the invention allows skilled artisans to easily use them in subsequent research and study. For example, it is possible to organize the expressed vaccinia virus polypeptides into an array for further analysis. The expressed polypeptide arrays can be screened in order to identify new vaccine and drug targets against microbial, neoplastic disease and the like, for example. The expressed polypeptides can be used to screen antibody libraries, to develop reagents, for functional proteomic studies, and the like. These steps can be rapidly accomplished at rates far exceeding traditional methods.

Adapter Technology

In addition to amplifying vaccinia virus polynucleotides of interest using TAP technology, the present invention also encompasses amplifying vaccinia virus polynucleotides using “adapter technology”. In some embodiments adapter technology is performed using a one-step PCR reaction. The term “adapter technology” as used herein relates to methods of cloning a desired polynucleotide into a vector by flanking a desired nucleic acid sequence, a vaccinia virus TAP fragment for example, with first and second adapter sequences. The resulting fragment can be contacted with the vector having sequences homologous to the first and second adapter sequences under conditions such that the nucleic acid fragment is incorporated into the vector by homologous recombination in vivo in a host cell. Accordingly, adapter technology allows for fast and enzymeless cloning of nucleic acid fragments into vectors and can also be used for forced cloning selection for successful transformation. Adapter technology is described in more detail in U.S. patent application Ser. No. 09/836,436, entitled “Fast and Enzymeless Cloning of Nucleic Acid Fragments”, U.S. patent application Ser. No. 10/125,789, entitled “Rapid and Enzymeless Cloning of Nucleic Acid Fragments”, and PCT Application No. PCTUS 02/12334, all of which are hereby incorporated by reference in their entirety.

The nucleic acid fragment can be incorporated into any vector using adaptor technology. In certain embodiments, the vector that the fragment is incorporated into can be, for example, a plasmid, a cosmid, a bacterial artificial chromosome (BAC), and the like. The plasmid can be CoE1, PR100, R2, pACYC, and the like. The vector can also include a functional selection marker. The functional selection marker can be, for example, a resistance gene for kanamycin, ampicillin, blasticidin, carbonicillin, tetracycline, chloramphenicol, and the like. The vector further can include a dysfunctional selection marker that lacks a critical element, and wherein the critical element is supplied by said nucleic acid fragment upon successful homologous recombination. The dysfunctional selection marker can be, for example, kanamycin resistance gene, ampicillin resistance gene, blasticidin resistance gene, carbonicillin resistance gene, tetracycline resistance gene, chloramphenicol resistance gene, and the like. Further, the dysfunctional selection marker can be, for example, a reporter gene, such as the lacZ gene, and the like.

The vector can include a negative selection element detrimental to host cell growth. The negative selection element can be disabled by said nucleic acid fragment upon successful homologous recombination. The negative selection element can be inducible. The negative selection element can be, for example, a mouse GATA-1 gene. The vector can include a dysfunctional selection marker and a negative selection element.

The host cell used in adapter technology can be a bacterium. The bacterium can be capable of in vivo recombination. Examples of bacterium include JC8679, TB1, DHα, DH5, HB101, JM101, JM109, LE392, and the like. The plasmid can be maintained in the host cell under the selection condition selecting for the functional selection marker.

The first and second adapters can be any length sufficient to bind to the homologous sequences of the vector such that the desired nucleic acid sequence is incorporated into the vector. The first and second adapter sequences can be, for example, at least 11 bp, 12 bp, 13, bp, 14 bp, 15 bp, 16 bp, 17 bp, 18 bp, 19 bp, 20 bp, 21 bp, 22 bp, 23 bp, 24 bp, 25 bp, 26 bp, 27 bp, 28 bp, 29 bp, 30 bp, 31 bp, 32 bp, 33 bp, 34 bp, 35 bp, 36 bp, 37 bp, 38 bp, 40 bp, 50 bp, 60 bp and the like. Furthermore, the first and second adapter sequences can be greater than 60 bp.

The first and second adapter sequences further can include a functional element. The functional element can include a promoter, a terminator, a nucleic acid fragment encoding a selection marker gene, a nucleic acid encoding a linker molecule, a nucleic acid fragment encoding a known protein, a fusion tag, a nucleic acid fragment encoding a portion of a selection marker gene, a nucleic acid fragment encoding a growth promoting protein, a nucleic acid fragment encoding a transcription factor, a nucleic acid fragment encoding an autofluorescent protein (e.g. GFP), and the like.

When the common sequences on both the 5′ and 3′ ends of the nucleic acid fragment are complimentary with terminal sequences in a linearized empty vector, and the fragment and linearized vector are introduced, by electroporation, for example, together into a host cell, they recombine resulting in a new expression vector with the fragment directionally inserted. In alternative embodiments the host cell can include the linearized empty vector so that only the nucleic acid fragment is introduced into the host cell. It should be noted that in alternative embodiments of the present invention the vector can be circularized, and as used herein a vector can be either linearized or circular. The host cell is converted into an expression vector through homologous recombination. In principle this approach can be applied generally as an alternative to conventional cloning methods.

A nucleic acid fragment having first and second adapter sequences can be generated by methods well known to those of skill in the art. For example, a gene of interest with known 5′ and 3′sequences undergoes PCR along with overlapping 5′ and 3′priming oligonucleotides. The priming oligonucleotides can be obtained by methods known in the art, including manufacture by commercial suppliers. A primary fragment with adapter sequences can be generated. The adapter sequences flanking the gene of interest can be homologous to sequences on a vector or to sequences from other 5′ or 3′fragments to be used in a subsequent PCR.

In some embodiments of the invention, a particular polynucleotide of interest from a vaccinia virus can be amplified with an adapter sequence on both the 3′ and 5′ ends. In other embodiments adapters can be attached to a plurality of polynucleotides, for example every coding region in the vaccinia virus genome. In certain embodiments adapters can make the desired coding regions transcriptionally active. Once incorporated into the desired vector, the vaccinia virus coding region can be rapidly replicated and expressed, such that a plurality of vaccinia virus's genes, for example every gene, is expressed.

Pluralities of expression products can be stored in libraries or arrays and can be assayed for their immunogenic properties as will be discussed below. While most embodiments relating to the assay methodologies are discussed in terms of TAP technology, all of the following assays can be used on adapter technology expression products as well. Once the appropriate assays are conducted on the adapter technology expression products, methods of developing vaccines can be utilized. While most of the embodiments relating to developing vaccines, discussed below, pertain to TAP technology, all of the vaccine embodiments can also be used with polypeptide libraries and arrays resulting from adapter technology.

Identifying Immunogenic Polypeptides

Libraries and arrays of polypeptides, prepared through TAP or adapter technology with subsequent expression can be useful in the development of polypeptide or nucleic acid subunit vaccines. DNA vaccines are effective vaccines that are inexpensive to manufacture, and can be widely distributed. It has been found that plasmid DNA, when injected into mice without being associated with any adjuvant, can generate antibody and CTL responses to viral antigens encoded by the plasmid DNA, and elicit protective immunity against viral infection (Ulmer at al., Science, 259:1745, 1993). Starting from this, there have been reported many research results regarding the induction of humoral and cellular immune responses resulting from the introduction of DNA vaccines containing various viral genes in animal models (Chow et al., J. Virol., 71:169, 1997; McClements et al., Proc. Natl. Acad. Sci. USA, 93:11414, 1996; Xiang et al., Virology, 199:132, 1994; Wang et al., Virology, 211:102, 1995; Lee et al., Vaccine, 17:473, 1999; Lee et al., J. Virol., 72:8430, 1998). As well, DNA delivery by electroporation techniques has been well-described (Heller et al., Expert Opin. Drug Deliv. 2(2): 1-14, 2005)

One of the most difficult tasks in developing a DNA vaccine (or any recombinant subunit vaccine) to a pathogen such as vaccinia virus, is the identification of antigens that can stimulate the most effective immune response against the pathogen, particularly when the genome of the organism is large.

A comprehensive means to accomplish this task, which is embodied by the present invention, is to obtain a plurality of polypeptides from the particular pathogen in the mode of a library or array. These polypeptides can be tested to determine their capability to evoke a humoral and/or a cell-mediated immune response. Polypeptides that evoke immunogenic responses can be tested individually or with other antigens for effectiveness as subunit vaccines. In addition, nucleic acids that encode identified antigenic polypeptides can be used alone or with other nucleic acids that encode antigens to develop a recombinant vaccine, such as a DNA vaccine, for the particular pathogen.

Vaccinia Virus Scanning

One embodiment of the invention, incorporates a Rapid High-Throughput Vaccine Antigen Scanning approach, using TAP Express, that is able to systematically screen and identify all, substantially all, or a subset all of the antigens in vaccinia virus that give rise to a humoral and cell-mediated immune response. The identification of the vaccinia virus antigens allows for the development of a highly specific subunit vaccine.

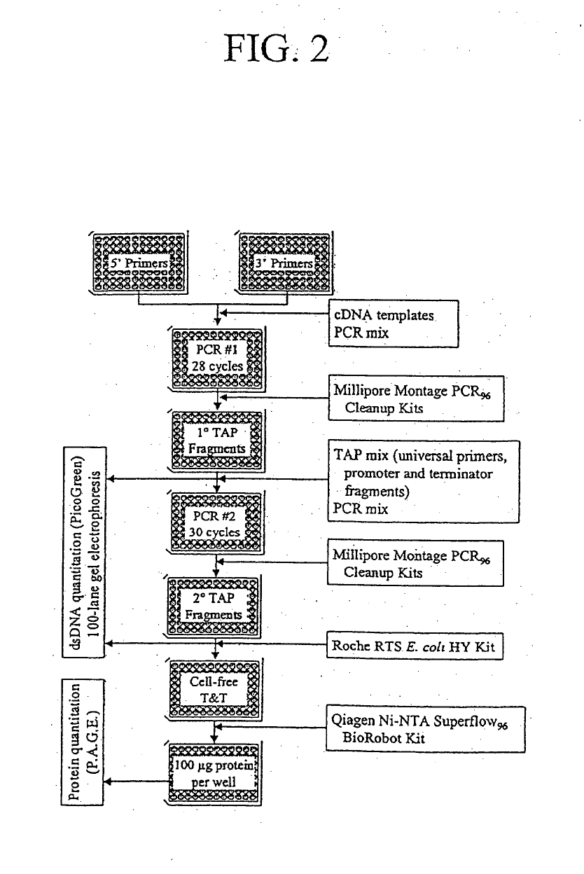

FIG. 2 illustrates a method for amplifying multiple vaccinia virus polynucleotides using TAP technology, expressing the gene products of the resultant TAP fragments, purifying, and quantifying the resulting polypeptides. FIG. 2 further illustrates a method of preparing polypeptides, which can be assayed to identify their ability to evoke a cell-mediated or humoral immune response.

In certain methods of developing a vaccinia virus vaccine, a plurality of vaccinia virus polynucleotides can be made transcriptionally active. In one embodiment, approximately 266 expression vectors, each encoding one of the 266 open reading frames from vaccinia virus genome can be made transcriptionally active using TAP technology. The present invention thus provides vaccinia virus polynucleotides including SEQ ID NOS: 52-72 that have been rendered transcriptionally active.

The resulting vaccinia virus TAP fragments of the present invention can be purified and expressed in vitro or in vivo according to any method known in the art. The expression products, which encompass SEQ ID NOS: 73-93, can be assayed to determine their ability to evoke a humoral and/or a cell-mediated immunogenic response. Polypeptides that are identified as capable of evoking an immune response can be used to develop polynucleotide or polypeptide subunit vaccines. The complete method will be described in more detail below.

According to one embodiment of the present invention, TAP fragments from vaccinia virus are used to generate a DNA array, and then, if desired, a protein array. In certain embodiments, primers are designed for every gene in the vaccinia virus genome. In another embodiment, designing the primers allows a skilled artisan to make any given vaccinia virus polynucleotide transcriptionally active using TAP technology. In yet another embodiment, coding regions, ORFs and other polynucleotide sequences of interest are ranked and the top vaccinia virus polynucleotides are made transcriptionally active using TAP technology.

As mentioned above, the custom PCR primers can be designed by using an automated system, such as a computerized robotics system. For example, in order to design custom primers for use in the TAP process, a robotic workstation can be interfaced with a dual Pentium III CPU (1.4 GHz) computer running the Linux operating system. In addition, a customized MySQL database can manage all the input sequence data from GenBank and from other sources. This database can track all the operations, samples and analytical data generated by the robot. In another embodiment, PCR primers, PCR products and polypeptides can be tracked by the database. For example, PCR primers, PCR products and polypeptides can be tracked by using bar coded 96-well plates. While the embodiments below discuss using 96-well plates in certain embodiments, those skilled in the art can appreciate that any sized well plate can be used. For example, the well plates can consist of about 48, about 96, about 144, about 192, about 240, about 288, about 336, about 384, about 432, about 480, about 576, about 672, about 768, about 864, about 960, about 1056, about 1152, about 1248, about 1344, about 1440, about 1536 or more wells. In addition to well plates, the PCR products and polypeptides can be tracked using any suitable receptacles, for example test tubes.

Custom oligonucleotide pairs of the present invention (SEQ ID NOS: 10-51), which are needed for the TAP PCR reactions, can be synthesized or obtained in order to perform the TAP technology. In one embodiment, the vaccinia virus genome sequence data and primer design software (e.g., Primer 3) can be used by the system to generate custom primer pairs for all, substantially all, or a subset of the genes in the vaccinia virus genome. The primers can be organized into arrays of about 48, about 96, about 144, about 192, about 240, about 288, about 336, about 384, about 432, about 480, about 576, about 672, about 768, about 864, about 960, about 1056, about 1152, about 1248, about 1344, about 1440, or about 1536 5′ primers and 3′ primers according to polynucleotide size and GC content, such PCR reaction conditions can be optimized on a plate by plate basis. The present invention further contemplates that sequences for each of the custom vaccinia virus primer pairs can be sent to an oligonucleotide synthesis provider (e.g., MWG Biotech, Inc, High Point, N.C.) where they can be synthesized. Synthesized primers can be organized and dispensed into bar-coded plates at a desired concentration, such as 100 pmole/μl, frozen and shipped to the practitioner. In one embodiment, 524 vaccinia virus-specific PCR primers, which are capable of amplifying each of the 266 vaccinia virus coding sequences are designed, generated, ordered, and organized.

After obtaining or generating the custom vaccinia virus PCR primers, the vaccinia virus polynucleotides of interest can be amplified. In one embodiment, the primers can be organized into arrays of 96 5′ primers and 96 3′primers according to polynucleotide size, and placed onto a robotic workstation. The robot can be programmed to generate a plate of about 48, about 96, about 144, about 192, about 240, about 288, about 336, about 384, about 432, about 480, about 576, about 672, about 768, about 864, about 960, about 1056, about 1152, about 1248, about 1344, about 1440, about 1536 PCR reactions by mixing the appropriate 5′ and 3′ primers with Taq polymerase and vaccinia virus genomic DNA. In addition to Taq, any thermally stable polymerase can be used in the PCR reactions. For example, Vent, Pfu, Tfl, Tth, and Tgo polymerases can be used. The robotic workstation can transfer the PCR reaction plate containing the mixed reagents to a PCR machine for amplification. In one embodiment, the robotic workstation can use a robotic arm to transfer the PCR reaction plate to the PCR machine.

The first TAP PCR procedure can be run for any number of cycles. In one embodiment, the PCR machine is run for about 20, 21, 22, 23, 24, 25, 26, 27, 28, 29, 30, 31, 32, 33, 34, 35, 36, 37, 38, 39, 40, or more cycles. The first TAP PCR reactions can be transferred robotically to a Millipore Montage 96-well cleanup kit, for example, when desired. Any method, kit or system can, however, be used to purify the PCR products from these reactions. According to one embodiment, a vacuum station of the robotic platform can carry out the purification step. In some embodiments, an aliquot of the resulting product can be transferred robotically to an analysis plate containing the Pico-Green fluorescent probe (Molecular Probes, Eugene, Oreg.) which reacts only with the dsDNA products. Depending on the number of wells, the plate can be transferred to an about 48, about 96, about 144, about 192, about 240, about 288, about 336, about 384, about 432, about 480, about 576, about 672, about 768, about 864, about 960, about 1056, about 1152, about 1248, about 1344, about 1440, about 1536 or more well fluorescent plate reader. The fluorescent signal can be compared to a standard curve to determine the amount of double stranded PCR product generated in this first PCR step. Persons with skill in the art can adjust the above methods in order to optimize their particular PCR reaction, should the need arise.

In addition to the first TAP PCR procedure, a second TAP PCR reaction can be performed to add at least one sequence that confers transcriptional activity to the primary TAP primary fragment. In one embodiment, a robot can be programmed to transfer an aliquot of each TAP primary fragment from the first TAP PCR reaction into a PCR reaction containing a promoter- and a terminator-containing primers. In a particular embodiment, the promoter can be a T7-his tag promoter sequence and the terminator can be a T7-his tag terminator sequence. Those with skill in the art can appreciate that any promoter or terminator sequence can be added to the primary transcript. In addition, any polynucleotide sequence that encodes a tag or linker allowing the expressed polypeptide to be detected or purified is also contemplated.

Like the first TAP PCR reaction, the second TAP PCR reaction can be run for any desired number of cycles. In one embodiment, the second TAP PCR reaction is run for about 20, 21, 22, 23, 24, 25, 26, 27, 28, 29, 30, 31, 32, 33, 34, 35, 36, 37, 38, 39, 40 cycles or more. Furthermore, any type of thermally stable polymerase can be used for the second TAP PCR reaction. In a particular embodiments the polymerase can be Taq. In some embodiments Vent, Pfu, Tfl, Tth, and Tgo polymerases can be used. The resulting TAP Express PCR fragments from the second PCR reaction can be cleaned by any kit, method or system. A particular kit that can be used to clean the resulting TAP fragments is a Millipore Montage 96-well cleanup kit. Additionally, as discussed above, the level of PCR product recovered can be determined using any detection agent, for example, Pico-Green.

The resulting TAP fragments can be expressed by using any method of gene expression. In one embodiment, the TAP fragments can be expressed using in vivo or in vitro (e.g. cell-free) systems. For example, the fragments can be directly transfected into any eukaryotic or prokaryotic cell for expression. Examples of eukaryotic cells that can be used for expression include mammalian, insect, yeast, and the like. An example of a prokaryotic cell expression system includes E. coli. The TAP fragments can also be expressed by a cell-free system. According to one embodiment of the invention, the resulting TAP fragments can be expressed in a high-throughput cell-free expression machine, such as, for example, the Roche RTS (Rapid Translation System) 100. In a further embodiment, the TAP fragments can be incubated in Roche RTS 100 system at 30° C. for 5 hours. A person with skill in the art can readily appreciate the utility in following the particular cell-free translation machine's instructions. If a T7-histadine promoter or terminator fragment is added to a primary transcript, translation of the TAP fragment can result in histidine tagged polypeptides, which can be purified as discussed below. As discussed herein, any tag can be used.

The expressed vaccinia virus polypeptides can be purified using any purification method for purifying expressed polypeptides. In one embodiment histidine tagged polypeptides can be purified with Qiagen nickel columns, such as Ni-NTA Superflow 96 Biorobot Kit. A person with skill in the art can readily appreciate the utility in following the instructions of the particular polypeptide purification system. Other methods that can be used to purify polypeptides include ultrafiltration, extraction, and chromatography.

The identity, quantity and purity of the purified Vaccinia polypeptides can be verified by SDS gel electrophoresis. According to one embodiment of the invention, MALDI-TOF MS (Matrix Assisted Laser Desorption/Ionization-Time of Flight Mass Spectrometry) can be employed to confirm the fidelity of the purified polypeptides. According to this embodiment, aliquots of each polypeptide (1-2 μg) can be aliquoted into about 48, about 96, about 144, about 192, about 240, about 288, about 336, about 384, about 432, about 480, about 576, about 672, about 768, about 864, about 960, about 1056, about 1152, about 1248, about 1344, about 1440, about 1536 or more well plates and digested with modified trypsin. The resulting material can be mixed with matrix (alpha-cyano-4-hydroxycinnamic acid (CHCA)) and spotted onto any target plate with a suitable number of spots, for example, 48, about 96, about 144, about 192, about 240, about 288, about 336, about 384, about 432, about 480, about 576, about 672, about 768, about 864, about 960, about 1056, about 1152, about 1248, about 1344, about 1440, about 1536 or more spots. In one embodiment, a 384-spot “anchor chip” target plate (Bruker Daltonics, Billerica, Mass.) can be used. The plate can be transferred to the sample stage of a Bruker Autoflex MALDI-TOF mass spectrometer. The spectrometer can be set up to automatically scan the plate and search the Mascot polypeptide database via the Internet. Accordingly, a very rapid verification system can verify purity, identity, and quantity in less than a day, for example, depending on the amount of polypeptides. Purified vaccinia virus polypeptides can be placed in libraries or organized into arrays for subsequent testing and analysis.

Humoral Immune Response

Use of the vaccinia virus polypeptide libraries and arrays prepared, for example, according the methods above (e.g. using TAP or adapter technology) can be used to identify antigenic targets of humoral immunity in vaccinia virus non-human animals and human patients. A humoral immune response relates to the generation of antibodies and their ability to bind to a particular antigen. In general, the humoral immune system uses white blood cells (B-cells), which have the ability to recognize antigens, to generate antibodies that are capable of binding to the antigens.

In one embodiment, the vaccinia virus polypeptides of the invention are generated according to the methods described above. In certain aspects of this embodiment additional polynucleotide sequences that encode linker molecules are added to the TAP primary fragment or the TAP expression fragment such that the expressed vaccinia virus polypeptides are fused to a linker molecule. As discussed previously, the term “linker molecule” encompasses molecules that are capable of immobilizing the polypeptides to a solid support.

In a particular embodiment, a vaccinia virus polynucleotide of interest is fused to a HA epitope tag such that the expressed product can include the vaccinia virus gene product fused to the HA epitope. In another embodiment, a vaccinia virus polynucleotide of interest is combined with a histidine coding sequence, such that the expressed product can include the vaccinia virus gene product and a 6×, 7×, 8×, 9×, or 10× histidine tag. In other embodiments a vaccinia virus polynucleotide is combined with a sequence that codes for a GST tag, fluorescent protein tag, or Flag tag. Using these methods it is possible to express and tag every Vaccinia virus polypeptide encoded by its genome. In another embodiment, the tagged vaccinia virus polypeptide can be attached to a solid support, such as a 96-well plate. The immobilize polypeptides can be contacted with an antiserum or other fluid containing antibodies from an animal that has been immunized with one or more antigens from a vaccinia virus. In one embodiment, ELISA and Western blot assays are performed in parallel to detect the presence of immunogenic vaccinia virus polypeptides.

As an example of an ELISA assay, tagged vaccinia virus polypeptides can be immobilized on a solid support, such as a 96-well plate. The immobilized vaccinia virus polypeptides are then incubated with serum from an animal that has been immunized with one or more antigens from a vaccinia virus. The reaction mixture can be washed to remove any unbound serum antibodies. The ability of the serum antibodies to bind to the bound vaccinia virus polypeptides can then be detected using any one of a number of methods. For example, enzyme linked secondary antibodies can be added to detect the presence of an antigen specific antibody. Any enzyme linked secondary antibody can be used in this invention, depending on the source of the serum. For example, if vaccinated mouse serum is used to provide the primary antibody, enzyme linked anti-mouse antibody can be used as a secondary antibody. Likewise if human serum is used to provide the primary antibody, enzyme linked anti-human serum can be used as a secondary enzyme.

Any suitable assay can be used to determine the amount of bound polypeptide specific antibody. Also, skilled artisans can develop the enzyme assay to determine the amount of polypeptide specific antibody that is bound. In one embodiment, the readout from an assay can show the presence of different levels of antibody in each of the 96 wells. For example, while some vaccinia virus polypeptides are not able to elicit any serum antibodies, other vaccinia virus polypeptides can elicit intermediate levels of antibodies, and some can elicit high antibody levels. In one embodiment, polypeptides that generate high antibody titers can be further researched to determine which polypeptides are present on the surface of the virus. In a particular embodiment of the invention vaccinia virus polypeptides that generate high antibody titers and that are located on the surface of the virus are candidates for use in the development of a subunit vaccinia virus vaccine.

In addition, serodiagnostic tests may be developed using antigens identified and characterized by these methods. That is, the peptide (epitopes) identified herein find use in detecting antibodies in serum from vaccinia infected or exposed organisms, animals or patients.

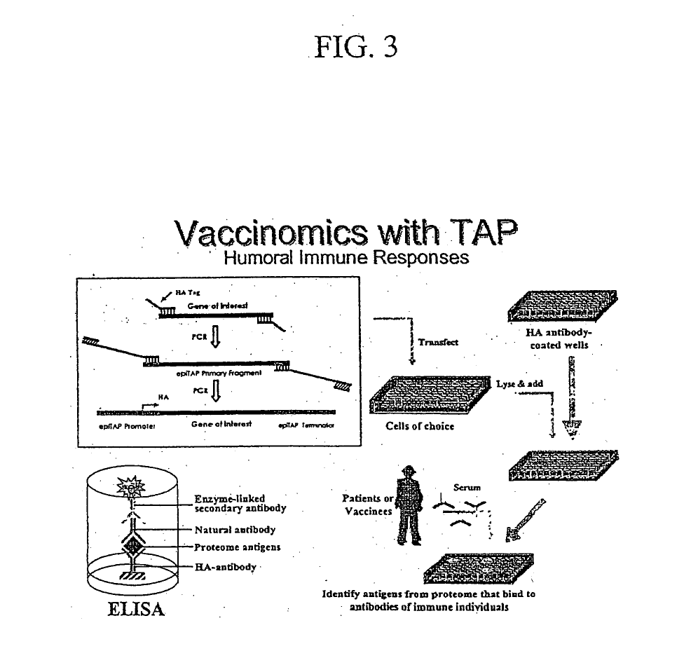

FIG. 3 demonstrates one embodiment of determining the humoral immune response generated by an array of polypeptides. One of skill in the art may deviate in certain details from those shown in FIG. 3. For example, the HA tag may be placed at either the C-terminal or N-terminal end of the polypeptide to insure that epitopes are not concealed due to binding to the plate. Instead of HA tagged polypeptides, a histidine tag can be used, and the polypeptides can be bound to nickel coated plates. For example a 6×, 7×, 8×, 9×, or 10× histidine tag can be used. Alternatively, histidine tagged polypeptides can be purified from either transfected cells or from the in vitro transcription translation system. Furthermore, purified vaccinia virus polypeptides can be attached non-specifically to polypeptide-absorbing plates such as Immulon plates, for example.

In one aspect of the present invention, highly immunogenic vaccinia virus antigens are detected by comparing the results of Western blotting analysis with ELISA. Western blotting and ELISA are two independent yet complementary methods that may be used to detect immunogenic vaccinia virus in qualitative and quantitative ways. Western blotting is often used to the examine the quality of a polypeptide or protein sample, including such parameters as purity, protein integrity, and degradation. Western blotting detects polypeptide in their denatured form. In one aspect of this embodiment, ELISA, which detects native polypeptides, is used to further examine Western-positive vaccinia virus polypeptides in a more quantitative fashion, to illustrate the strength of the vaccinia virus epitope's immunogenicity.

Cell-Mediated Immune Response

Use of the TAP-expressed vaccinia virus polypeptide libraries and arrays prepared according the methods above (e.g. using TAP or adapter technology) can also be exploited to identify the highly immunogenic targets of cell-mediated immunity in vaccinia virus vaccinated non-human animals. In contrast to a humoral immune response, where an antibody binds directly binding to an antigen, a cell-mediated immune response relates to T-cells binding to the surface of other cells that display the antigen. When certain T-cells come into contact with a presented antigen, they produce and release cytokines such as interferon-γ (IFN-γ) or Tumor Necrosis Factor-alpha (TNF-α). Cytokines are cellular signals that can alter the behavior or properties of another cell. For example, cytokines may inhibit viral replication, induce increased expression of MHC class I and peptide transporter molecules in infected cells, or activate macrophages. Accordingly, cytokines released by T-cells, associated with the binding to an antigen, can be used to identify and detect T-cell/antigen interactions.

Some cells have MHC molecules on their membranes to present antigens to T-cells. Efficient T-cell function relies on proper recognition of the MHC-antigen complex. There are two types of MHC molecules: Class I and Class II. The two different classes of MHC molecules bind peptides from different sources inside the cell for presentation at the cell surface to different classes of T-cells. Any T-cell can be used in the present invention, and include for example both CD4⁺ and CD8⁺ T-cells. CD8⁺ cells (cytotoxic T-cells) bind epitopes that are part of class I MHC molecules. CD4⁺ T-Cells, which includes inflammatory CD4 T-cells and helper CD4 T-cells, bind epitopes that are part of class II MHC molecules. Only specialized antigen-presenting cells express class II molecules.

There are three main types of antigen-presenting cells: B cells, macrophages and dendritic cells. Each of these cell types is specialized to process and present antigens from different sources to T-cells, and two of them, the macrophages and the B cells, are also the targets of subsequent actions of armed effector T-cells. These three cell types can express the specialized co-stimulatory molecules that enable them to activate naïve T-cells, although macrophages and B cells express those molecules only when suitably activated by infection.

Embodiments of the present invention relate to detecting vaccinia virus polypeptides capable of evoking a cell-mediated immune response in order to identify potential candidates for use in a subunit vaccine or other pharmaceutical composition. According to one method of detecting a cell-mediated immune response, a vaccinia virus polypeptide is delivered into an antigen-presenting cell where it can be presented in a manner that is recognized by antigen specific T-cells. In another embodiment of the invention, a transcriptionally active gene can be delivered into an antigen-presenting cell where expressed and presented in a manner that can be recognized by an antigen specific T-cells. Vaccinia virus antigen specific T-cells can be acquired from numerous sources. For example, animals that have been immunized with one or more antigens from vaccinia virus virus are a good source of antigen specific T-cells. Alternatively, a human volunteer immunized with vaccinia virus can be a source of antigen specific T-cells.

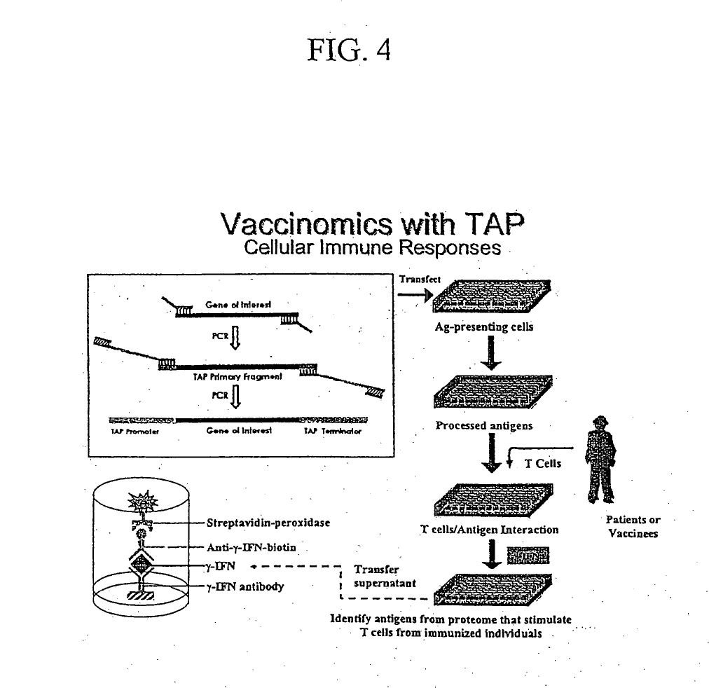

FIG. 4 demonstrates one embodiment of determining the cell-mediated immune response generated by an array of polypeptides. One of skill in the art may deviate in certain details from those shown in FIG. 4.

In order to test the ability of vaccinia virus polypeptides to elicit a cell mediated response, a plurality of vaccinia virus polynucleotides can be amplified and made transcriptionally active using TAP technology. In one embodiment about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 266 vaccinia virus polynucleotides are made transcriptionally active using TAP technology.

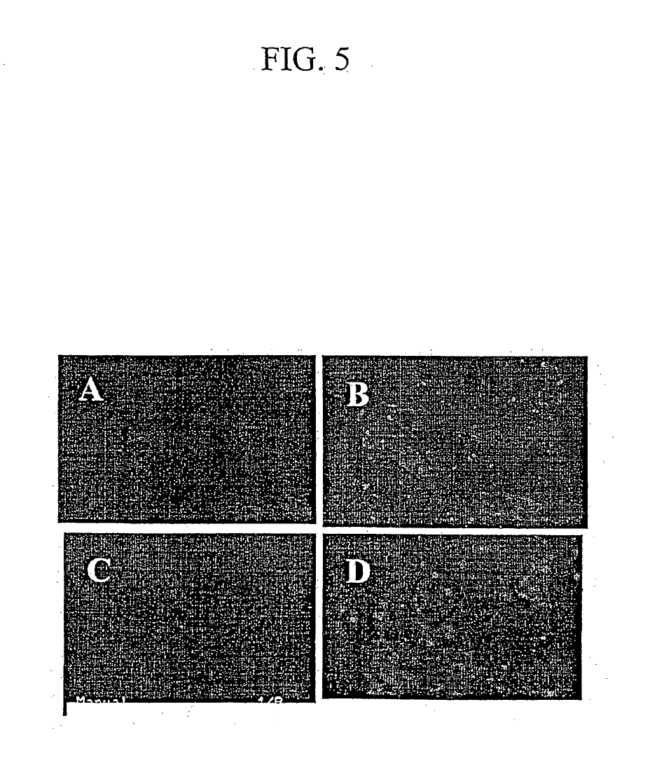

In one embodiment, transcriptionally active vaccinia virus polynucleotides can be transfected into an antigen-presenting cell and expressed within the cell. In another embodiment, instead of transfecting the genes into an antigen-presenting cell, the vaccinia virus TAP fragments can be expressed in an in vivo or in vitro (cell-free) expression system and the expressed polypeptide can be delivered into the antigen-presenting cell. The polypeptide can be delivered into the antigen-presenting cell according to any method. In one embodiment, the polypeptide can be delivered using the technology described in U.S. patent application Ser. No. 09/738,046, entitled “Intracellular Protein Delivery Reagent” and U.S. patent application Ser. No. 10/141,535, entitled “Intracellular Protein Delivery Compositions and Methods of Use,” both of which are hereby incorporated by reference in their entirety. The reagents described therein are is capable of delivering any type of polypeptide into any type of cell. Furthermore, the results of FIG. 5 demonstrate that dendritic cells can present antigens to T-Cells supplied from an immunized host after antigenic polypeptides were delivered to the dendritic cells with reagents from the above mentioned applications.

In certain embodiments of the invention, reagents used to deliver polypeptides into cultured cells can be a cationic lipid formulation. In one embodiment, these reagents can deliver fluorescently labeled antibodies, high and low molecular weight dextrans, phycoerythrin-BSA, caspase 3, caspase 8, granzyme B, and β-galactosidase into the cytoplasm of a variety of different adherent and suspension cells. Caspases delivered to cells with are functional, since they can be shown to send cells into apoptosis. In one embodiment, vaccinia virus polypeptides are delivered into dendritic cells using these reagents.

Detecting a T-cell's ability to bind to an antigen-presenting cell, after the antigen-presenting cell has processed a particular polypeptide, is useful in determining whether the particular polypeptide evokes a cell-mediated immune response. Once a particular polypeptide is delivered into or expressed in the antigen-presenting cell, an assay can be performed to identify T-cell interaction with the MHC-antigen complex. In one embodiment, it can be determined if T-cells obtained from an animal that was immunized with vaccinia virus can bind to a particular antigen presented by an antigen-presenting cell. For example, an ELIspot assay (Enzyme-Linked Immuno spotting; ELIspot) assay can be performed to identify antigen specific T-cells. Similar immunoassays can be performed to identify vaccinia virus antigens (presented by an antigen-presenting cells) that stimulate T-cells from vaccinia virus immunized individuals.

One method of detecting a T-cell/antigen interaction is to measure the amount of a particular cytokine released by the T-cell when it interacts with a MHC-antigen complex. The skilled artisan can appreciate that other cellular signals can be used to indicate a cell-mediated immune response. In one embodiment, the levels of IFN-γ released by T-cells can indicate whether a particular peptide is capable of evoking a cell-mediated immune response. In a particular embodiment, an antibody specific for IFN-γ can be coated onto a solid support. Unbound antibodies can be washed away and IFN-γ obtained from the supernatant containing T-cells plus antigen-presenting cells or antigen transduced antigen-presenting cells, can be added to the wells. A biotinylated secondary antibody specific for IFN-γ can be added. Excess secondary antibody can be removed and Streptavidin-Peroxidase can be added to the mixture. Streptavidin-Peroxidase is capable of binding to the biotinylated antibody to complete the four-member immunoassay “sandwich.” Excess or unbound Streptavidin-Peroxidase is easily removed from the mixture. In order to detect amount of bound Streptavidin-Peroxidase, a substrate solution can be added which reacts with the Streptavidin-Peroxidase to produce color. The intensity of the colored product is directly proportional to the concentration of IFN-γ present in the T-cell/antigen-presenting cell supernatant. Kits for performing these types of immunoassay are readily available from many commercial suppliers or the necessary reagents composing such kits can be purchased separately or produced in-house. In one embodiment, processed and presented vaccinia virus polypeptide that evokes T-cells to produce a high level of IFN-γ can be considered a strong candidate for use in developing a subunit vaccine.

Those with skill in the art will appreciate that other methods can be used to detect T-cell/Antigen interactions. These methods include bead based assays, flow-based assays, RT-PCR based assays, cytokine ELISAs, lymphoproliferation assays, cytotoxic T cell assays, or any other assay that can detect the interaction of a T-cell with a responder cell (e.g. macrophage).

Developing a Subunit Vaccine, Pharmaceutical Composition, or Immunogenic Composition

A particular vaccinia virus polypeptide that has been identified to elicit either a humoral or cell-mediated immune response, can be further explored to determine its ability to be used in a subunit vaccine, pharmaceutical composition, or immunogenic composition. The terms “subunit vaccine,” “DNA vaccine,” “recombinant vaccine” and “immunogenic composition” encompass vaccines that are comprised of polypeptides, nucleic acids or a combination of both. Further exploration of a vaccinia virus polypeptide vaccine candidate includes testing the vaccinia virus polypeptide or nucleic acid encoding the vaccinia virus polypeptide in a large percentage of patients. In a particular embodiment, surface antigens can be studied closely because of the likelihood that they can inhibit virus infectivity. In one embodiment, every polypeptide encoded by the vaccinia virus genome is assayed to determine its immunogenic effect. Polypeptides that elicit an immune response, whether cell-mediated or humoral, can be more closely studied to determine potential use alone or in conjunction with other polypeptides and genes as a subunit vaccine, pharmaceutical composition, or immunogenic composition. Suitable methodologies for electing and detecting an immune response are well established in the art.

Uses of Vaccine Compositions

As noted previously, the present invention provides peptide immunogens and nucleic acids encoding the immunogens. As such, the present invention also provides methods of using the immunogens to generate an immune response in a mammalian host.

Methods of generating immune responses in a host are known in the art. However, according to the present invention, the method includes administering to the host an immunogenic composition. The immunogenic composition includes at least one nucleic acid selected from SEQ ID NO: 52-72. In addition, fragments of these sequences can be used. Also, it should be noted that combinations of these sequences may be used to generate an immune response against vaccinia. When using nucleic acids to generate an immune response the nucleic acids preferably encode peptides found in SEQ ID NO: 73-93. In addition, fragments of these sequences can be used. Also, combinations of these sequences can be used.

When combinations of the above immunogenic compositions are to be used at least 2, 3, 4 or 5 or more of the nucleic acids or fragments thereof can be combined to generate an immunogenic composition. Any combination of the nucleic acids finds use in this method.

Also, methods of generating an immune response include administering to the host at least one peptide selected from the peptides found in SEQ ID NO: 73-93. In addition, fragments of these sequences can be used. Also, it should be noted that combinations of these sequences may be used to generate an immune response against vaccinia. When combinations of the above immunogenic compositions are to be used at least 2, 3, 4 or 5 or more of the peptides or fragments thereof can be combined to generate an immunogenic composition. Any combination of screened nucleic acids finds use in this method.

Kits

Various nucleic acids and peptides have been identified that generate an immune response. As such, the nucleic acids and peptides find use in kits. The kits of the invention are useful for a variety of applications including combining reagents necessary for producing vaccine compositions. Such vaccine compositions include the polypeptides and polynucleotides described herein as well as carriers, diluents and other pharmaceutically acceptable carriers. It should be noted, as described above, that the kits may include fragments of the nucleic acids or peptides described herein as well as combinations of the nucleic acids and/or peptides described herein. Preferably the kits include at least 2, 3, 5, 10, 15, 20, 25, 30 or more nucleic acids or peptides described herein. Any combination of the nucleic acids or peptides can be used. In addition, the kits may include adjuvants. In addition, the kits may include instructions for preparing and administering the vaccines.

In addition, the kits of the invention find use as diagnostic kits. In particular, the kits find use as serodiagnostic kits. As such, the kits include at least one peptide as described herein. Preferably, however, the kits include a plurality of peptides, such as at least 2, 3, 5, 10, 15 or 20 or more peptides for diagnosis of vaccinia infection or exposure of an organism, animal or patient.

In some embodiments, the nucleic acids encoding the polypeptides find use in diagnostic kits. The nucleic acids encoding the antigenic peptides find use as probes to detect complementary nucleic acids of vaccinia. However, in an alternative embodiment the kits include the polypeptides produced from the in vitro transcription-translation reaction find use in detecting antibodies from an organism, animal or patient exposed to Vaccinia.

### EXAMPLES

### Example 1

### Procedure for Generating Histidine Tagged TAP Express Fragments

A detailed procedure that was used to produce tagged T7-TAP Express fragments is as follows: 96 different genes were amplified from a mixture of plasmid templates. A first PCR reaction was run with customized 5′ and 3′ primers. The 5′primers contained between 43-48 bases. In particular, the T-7-His TAP ends contained 28 bases while the gene-specific component contained between 15-20 bases. The 3′primers contained between 45-50 bases. Specifically, the T7-terminator TAP ends contained 30 bases while the gene specific component contained between 15-20 bases. The reaction temperature and times for the first PCR reaction were: 94° C. for 2 minutes, followed by 28 cycles of: 94° C. for 20 seconds, 58° C. for 35 seconds, and 70° C. for 2 minutes (for genes that contained more than 2 kb, 1 minute was added for each kb).

After the first PCR reaction was performed, an aliquot of each PCR reaction from the previous step was transferred into a PCR reaction containing the T7-histidine promoter fragment and T7 terminator fragment. The T7 promoter primer contained 25 bases, while the T7-promoter-His tag fragment contained a 104 base EcoRV/BglII fragment. The T7-terminator fragment was a 74 base oligonucleotide. The reaction temperature and times for the second PCR reaction were: 94° C. for 2 minutes, followed by 30 cycles of: 94° C. for 20 seconds, 60° C. for 35 seconds, and 70° C. for 2 minuets (for genes that contained more than 2 kb, 1 minute was added for each kb).

### Example 2

### Using the Vaccinia Virus Proteome to Identify Antigenic Targets of Humoral Immunity in Vaccinia Virus Vaccinated Mice and Humans

The following is a method used to systematically screen and identify from amongst the coding regions in the vaccinia virus genome, antigens that give rise to a protective humoral immune response. Initially, three hundred vaccinia virus genes were synthesized by TAP and ˜100 proteins were translated and purified in arrays, as described below.

TAP PCR

The PCR reactions were performed such that a nucleotide sequence encoding an His tag was fused to these amplified transcriptionally active genes. The resulting His tagged TAP fragments were expressed to produce vaccinia virus polypeptides containing the His tag

The detailed procedure that was used to produce tagged T7-TAP Express fragments is as follows: groups of 96 vaccinia virus polynucleotide sequences were amplified from vaccinia virus genomic DNA. A first PCR reaction was performed using customized 5′ and 3′primers, as shown in Table 1 (SEQ ID NOS: 10 and 11; 12 and 13; 14 and 15; 16 and 17; 18 and 19; 20 and 21; 22 and 23; 24 and 25; 26 and 27; 28 and 29; 30 and 31; 32 and 33; 34 and 35; 36 and 37; 38 and 39; 40 and 41; 42 and 43; 44 and 45; 46 and 47; 48 and 49, 50 and 51). The 5′primers contained between 43-48 bases. In particular, the T-7-His TAP ends contained 28 bases while the gene-specific component contained between 15-20 bases. The 3′primers contained between 45-50 bases. Specifically, the T7-terminator TAP ends contained 30 bases while the gene specific component contained between 15-20 bases.

TABLE 1

Immunogenic Vaccinia Virus Polypeptides: Primers,
Polynucleotide Sequences, and Amino Acid Sequences

GenBank protein_id: AAR17901
Locus: VACAC2_230 double-stranded RNA binding protein
5′ primer:
GAAGGAGATATACCATGCATCATCATCATCATCATATGTCTAAGATCTAT (SEQ ID NO:10)
3′ primer:
TGATGATGAGAACCCCCCCCTCAGAATCTAATGATGACGT (SEQ ID NO:11)
Polynucleotide sequence:
ATGTCTAAGATCTATATTGACGAGCGTTCTGACGCAGAGATTGTGTGTGCGGCTATTAAAAA
CATTGGAATCGAAGGAGCTACTGCTGCACAACTAACTAGACAACTTAATATGGAGAAGCGAG
AAGTTAATAAAGCTCTGTACGATCTTCAACGTAGTGCTATGGTGTACAGCTCCGACGATATT
CCTCCTCGTTGGTTTATGACAACGGAGGCGGATAAGCCGGATGCTGATGTTATGGCTGACGC
CATAATAGATGATGTATCCCGCGAAAAATCAATGAGAGAGGATCATAAGTCTTTTGATGATG
TTATTCCGGCTAAAAAAATTATTGATTGGAAAGATGCTAACCCTGTCACCATTATTAATGAG
TACTGCCAAATAACTAAGAGAGATTGGTCTTTTCGTATTGAATCAGTTGGGCCTAGTAACTC
TCCTACATTTTATGCCTGTGTAGATATCGACGGAAGAGTATTCGATAAGGCCGATGGAAAAT
CTAAACGAGATGCTAAAAATAATGCAGCTAAATTGGCTGTAGATAAACTTCTTGGGTACGTC
ATCATTAGATTCTGA 572 BP (SEQ ID NO:52)
Amino acid sequence:
MSKIYIDERSDAEIVCAAIKNIGIEGATAAQLTRQLNMEKREVNKALYDLQRSAMVYSSDDI
PPRWFMTTEADKPDADVMADAIIDDVSREKSMREDHKSFDDVIPAKKIIDWKDANPVTIINE
YCQITKRDWSFRIESVGPSNSPTFYACVDIDGRVFDKADGKSKRDAKNNAAKLAVDKLLGYV
IIRF (SEQ ID NO:73) 21.0 kDa
GenBank protein_id: AAR17976
Locus:VACAC2_144 phosphorylated IMV membrane protein
5′ primer:
GAAGGAGATATACCATGCATCATCATCATCATCATATGGACATGATGCTT (SEQ ID NO:12)
3′ primer:
TGATGATGAGAACCCCCCCCTTAGTTCACGGAAATATCGC (SEQ ID NO:13)
Polynucleotide sequence:
ATGGACATGATGCTTATGATTGGAAATTATTTTTCCGGCGTGCTAATCGCTGGAATCATTCT
TTTGATTCTTTCGTGTATCTTCGCCTTTATTGACTTTAGTAAGTCTACCAGTCCCACTCGTA
CATGGAAAGTATTGAGTATTATGGCGTTTATACTTGGTATTATTATCACAGTCGGAATGCTA
ATTTATTCTATGTGGGGAAAGCACTGCGCGCCCCACAGAGTTAGCGGAGTCATTCACACCAA
TCATAGCGATATTTCCGTGAACTAA 272 BP (SEQ ID NO:53)
Amino acid sequence:
MDMMLMIGNYFSGVLIAGIILLILSCIFAFIDFSKSTSPTRTWKVLSIMAFILGIIITVGML
IYSMWGKHCAPHRVSGVIHTNHSDISVN (SEQ ID NO:74) 10.0 kDa
GenBank protein_id: AAR17845
Locus:VACAC2_011 VACAC2_011
5′ primer:
GAAGGAGATATACCATGCATCATCATCATCATCATATGAATTTACAGAAA (SEQ ID NO:14)
3′ primer:
TGATGATGAGAACCCCCCCCCTACTTGTACGGTAGCGACG (SEQ ID NO:15)
Polynucleotide sequence:
ATGAATTTACAGAAATTATCTCTGGCTATATATCTTACTGCGACATGTTCGTGGTGTTATGA
AACATGCATAAGAAAAACTGCGTTGTATCATGACATTCAATTGGAGCATGTAGAAGACAATA
AAGATAGTGTAGCGTCGCTACCGTACAAGTAG 155 BP (SEQ ID NO:54)
Amino acid sequence:
MNLQKLSLAIYLTATCSWCYETCIRKTALYHDIQLEHVEDNKDSVASLPYK (SEQ ID NO:75)
5.7 kDa
GenBank protein_id: AAR17883
Locus:VACAC2_051 dUTPase
5′ primer:
GAAGGAGATATACCATGCATCATCATCATCATCATATGTTCAACATGAAAT (SEQ ID NO:16)
3′ primer:
TGATGATGAGAACCCCCCCCTTATCTAAGTCCTGTTGATC (SEQ ID NO:17)
Polynucleotide sequence:
ATGTTCAACATGAATATTAACTCACCAGTTAGATTTGTTAAGGAAACTAACAGAGCTAAATC
TCCTACTAGGCAATCGCCGGGTGCTGCCGGATATGATTTGTATAGCGCTTACGATTATACTA
TCCCTCCAGGAGAAAGACAGTTAATTAAGACAGATATTAGTATGTCCATGCCTAAGATTTGC
TATGGTAGAATAGCTCCTAGGTCTGGTCTGTCACTAAAAGGCATTGATATAGGAGGTGGTGT
AATAGACGAAGATTATAGGGGAAACATAGGAGTCATTCTTATTAATAATGGAAAATGTACGT
TTAATGTAAATACTGGAGATAGAATAGCTCAGCTAATCTATCAACGTATATATTATCCAGAA
CTGGAAGAAGTACAATCTCTAGATAGTACAAATAGAGGAGATCAAGGGTTTGGATCAACAGG
ACTTAGATAA 443 BP (SEQ ID NO:55)
Amino acid sequence:
MFNMNINSPVRFVKETNRAKSPTRQSPGAAGYDLYSAYDYTIPPGERQLIKTDISMSMPKIC
YGRIAPRSGLSLKGIDIGGGVIDEDYRGNIGVILINNGKCTFNVNTGDRIAQLIYQRIYYPE
LEEVQSLDSTNRGDQGFGSTGLR (SEQ ID NO:76) 16.2 kDa
GenBank protein_id: AAR17973
Locus:VACAC2_141 VACAC2_141
5′ primer:
GAAGGAGATATACCATGCATCATCATCATCATCATATGACGACCGTACCA (SEQ ID NO:18)
3′ primer:
TGATGATGAGAACCCCCCCCTTAAATAATTTTAATTCGTT (SEQ ID NO:19)
Polynucleotide sequence:
ATGACGACCGTACCAGTGACGGATATACAAAACGATTTAATTACAGAGTTTTCAGAAGATAA
TTATCCATCTAACAAAAATTATGAAATAACTCTTCGTCAAATGTCTATTCTAACTCACGTTA
ACAACGTGGTAGATAGAGAACATAATGCCGCCGTAGTGTCATCTCCAGAGGAAATATCCTCA
CAACTTAATGAAGATCTATTTCCAGATGATGATTCACCGGCCACTATTATCGAACGAGTACA
ACCTCATACTACTATTATTGACGATACTCCACCTCCTACGTTTCGTAGAGAGTTATTGATAT
CGGAACAACGTCAACAACGAGAAAAAAGATTTAATATTACAGTATCGAAAAATGCTGAAGCA
ATAATGGAATCTAGATCTATGATAACTTCTATGCCAACACAAACACCATCCTTGGGAGTAGT
TTATGATAAAGATAAAAGAATTCAGATGTTAGAGGATGAAGTGGTTAATCTTAGAAATCAAC
GATCTAATACAAAATCATCTGATAATTTAGATAATTTTACCAAAATACTATTTGGTAAGACT
CCGTACAAATCAACAGAAGTTAATAAGCGTATAGCCATCGTTAATTATGCAAATTTGAACGG
GTCTCCCTTATCAGTCGAGGACTTGGATGTTTGTTCAGAGGATGAAATAGATAGAATCTATA
AAACGATTAAACAATATCACGAAAGTAGAAAACGAAAAATTATCGTCACTAACGTGATTATT
ATTGTCATAAACATTATCGAGCAGGCATTGCTAAAACTCGGATTTGAAGAAATCAAAGGACT
GAGTACCGATATCACTTCAGAAATTATCGATGTGGAGATCGGAGATGACTGCGATGCTGTAG
CATCTAAACTAGGAATCGGTAACAGTCCGGTTCTTAATATTGTATTGTTTATACTCAAGATA
TTCGTTAAACGAATTAAAATTATTTAA 956 BP (SEQ ID NO:56)
Amino acid sequence:
MTTVPVTDIQNDLITEFSEDNYPSNKNYEITLRQMSILTHVNNVVDREHNAAVVSSPEEISS
QLNEDLFPDDDSPATIIERVQPHTTIIDDTPPPTFRRELLISEQRQQREKRFNITVSKNAEA
IMESRSMITSMPTQTPSLGVVYDKDKRIQMLEDEVVNLRNQRSNTKSSDNLDNFTKILFGKT
PYKSTEVNKRIAIVNYANLNGSPLSVEDLDVCSEDEIDRIYKTIKQYHESRKRKIIVTNVII
IVINIIEQALLKLGFEEIKGLSTDITSEIIDVEIGDDCDAVASKLGIGNSPVLNIVLFILKI
FVKRIKII (SEQ ID NO:77) 35.1 kDa
GenBank protein_id: AAR17993
Locus: VACAC2_161 IMV surface protein
5′ primer:
GAAGGAGATATACCATGCATCATCATCATCATCATATGGACGGAACTCTT (SEQ ID NO:20)
3′ primer:
TGATGATGAGAACCCCCCCCTTACTCATATGGACGCCGTC (SEQ ID NO:21)
Polynucleotide sequence:
ATGGACGGAACTCTTTTCCCCGGAGATGACGATCTTGCAATTCCAGCAACTGAATTTTTTTC
TACAAAGGCTGCTAAAAAGCCAGAGGCTAAACGCGAAGCAATTGTTAAAGCCGATGAAGACG
ACAATGAGGAAACTCTCAAACAACGGCTAACTAATTTGGAAAAAAAGATTACTAATGTAACA
ACAAAGTTTGAACAAATAGAAAAGTGTTGTAAACGCAACGATGAAGTTCTATTTAGGTTGGA
AAATCACGCTGAAACTCTAAGAGCGGCTATGATATCTCTGGCTAAAAAGATTGATGTTCAGA
CTGGACGGCGTCCATATGAGTAA 332 BP (SEQ ID NO:57)
Amino acid sequence:
MDGTLFPGDDDLAIPATEFFSTKAAKKPEAKREAIVKADEDDNEETLKQRLTNLEKKITNVT
TKFEQIEKCCKRNDEVLFRLENHAETLRAAMISLAKKIDVQTGRRPYE (SEQ ID NO: 78)
12.2 kDa
GenBank protein_id: AAR17906
Locus:VACAC2_074 myristylprotein
5′ primer:
GAAGGAGATATACCATGCATCATCATCATCATCATATGGGAACTGCTGCA (SEQ ID NO:22)
3′ primer:
TGATGATGAGAACCCCCCCCTTATATATTTAGTTTATCTT (SEQ ID NO:23)
Polynucleotide sequence:
ATGGGAACTGCTGCAACAATTCAGACTCCCACCAAATTAATGAATAAAGAAAATGCAGAAAT
GATTTTGGAAAAAATTGTTGATCATATAGTTATGTATATTAGTGACGAATCAAGTGATTCAG
AAAATAATCCTGAATATATTGATTTTCGTAACAGATACGAAGACTATAGATCTCTCATTATA
AAAAGTGATCACGAGTTTGTAAAGCTATGTAAAAATCATGCGGAGAAAAGTTCTCCAGAAAC
GCAACAAATGATTATCAAACACATATACGAACAATATCTTATTCCAGTATCTGAAGTACTAT
TAAAACCTATAATGTCCATGGGTGACATAATTACATATAACGGATGTAAAGACAATGAATGG
ATGCTAGAACAACTCTCTACCCTAAACTTTAACAATCTCCGCACATGGAACTCATGTAGCAT
AGGCAATGTAACGCGTCTGTTTTATACATTTTTTAGTTATCTGATGAAAGATAAACTAAATA
TATAA 500 BP (SEQ ID NO:58)
Amino acid sequence:
MGTAATIQTPTKLMNKENAEMILEKIVDHIVMYISDESSDSENNPEYIDFRNRYEDYRSLII
KSDHEFVKLCKNHAEKSSPETQQMIIKHIYEQYLIPVSEVLLKPIMSMGDIITYNGCKDNEW
MLEQLSTLNFNNLRTWNSCSIGNVTRLFYTFFSYLMKDKLNI (SEQ ID NO:79)
18.3 kDa
GenBank protein_id: AAR17944
Locus:VACAC2_112 IMV heparin binding surface protein
5′ primer:
GAAGGAGATATACCATGCATCATCATCATCATCATATGGCGGCGGTGAAA (SEQ ID NO:24)
3′ primer:
TGATGATGAGAACCCCCCCCTTAGATAAATGCGGTAACGA (SEQ ID NO:25)
Polynucleotide sequence:
ATGGCGGCGGTGAAAACTCCTGTTATTGTTGTGCCAGTTATTGATAGACCTCCATCAGAAAC
ATTTCCTAATGTTCATGAGCATATTAATGATCAGAAGTTCGATGATGTAAAGGACAACGAAG
TTATGCCAGAAAAAAGAAATGTTGTGGTAGTCAAGGATGATCCAGATCATTACAAGGATTAT
GCGTTTATACAGTGGACTGGAGGAAACATTAGAAATGATGACAAGTATACTCACTTCTTTTC
AGGGTTTTGTAACACTATGTGTACAGAGGAAACGAAAAGAAATATCGCTAGACATTTAGCCC
TATGGGATTCTAATTTTTTTACCGAGTTAGAAAATAAAAAGGTAGAATATGTAGTTATTGTA
GAAAACGATAACGTTATTGAGGATATTACGTTTCTTCGTCCCGTCTTGAAGGCAATGCATGA
CAAAAAAATAGATATCCTACAGATGAGAGAAATTATTACAGGCAATAAAGTTAAAACCGAGC
TTGTAATGGACAAAAATCATGCCATATTCACATATACAGGAGGGTATGATGTTAGCTTATCA
GCCTATATTATTAGAGTTACTACGGCGCTGAACATCGTAGATGAAATTATAAAGTCTGGAGG
TCTATCATCGGGATTTTATTTTGAAATAGCCAGAATCGAAAACGAAATGAAGATCAATAGGC
AGATACTGGATAACGCCGCCAAATATGTAGAACACGATCCCCGACTTGTTGCAGAACACCGC
TTCGAAAACATGAAACCGAATTTTTGGTCTAGAATAGGAACGGCAGCTGCTAAACGTTATCC
AGGAGTTATGTACGCGTTTACTACTCCACTGATTTCATTTTTTGGATTGTTTGATATTAATG
TTATAGGTTTGATTGTAATTTTGTTTATTATGTTTATGCTCATCTTTAACGTTAAATCTAAG
CTGTTATGGTTCCTTACAGGAACATTCGTTACCGCATTTATCTAA 974 BP
(SEQ ID NO:59)
Amino acid sequence:
MAAVKTPVIVVPVIDRPPSETFPNVHEHINDQKFDDVKDNEVMPEKRNVVVVKDDPDHYKDY
AFIQWTGGNIRNDDKYTHFFSGFCNTMCTEETKRNIARHLALWDSNFFTELENKKVEYVVIV
ENDNVIEDITFLRPVLKAMHDKKIDILQMREIITGNKVKTELVMDKNHAIFTYTGGYDVSLS
AYIIRVTTALNIVDEIIKSGGLSSGFYFEIARIENEMKINRQILDNAAKYVEHDPRLVAEHR
FENMKPNFWSRIGTAAAKRYPGVMYAFTTPLISFFGLFDINVIGLIVILFIMFMLIFNVKSK
LLWFLTGTFVTAFI (SEQ ID NO:80) 35.7 kDa
GenBank protein_id: AAR17933
Locus:VACAC2_101 VACAC2_101
5′ primer:
GAAGGAGATATACCATGCATCATCATCATCATCATATGAATACCCGTACC (SEQ ID NO:26)
3′ primer:
TGATGATGAGAACCCCCCCCTTATACTATAATAGTACTAG (SEQ ID NO:27)
Polynucleotide sequence:
ATGAATACCCGTACCGATGTTACAAACGATAATATAGACAAAAATCCAACCAAACGAGGTGA
TAGAAATATACCAGGAAGAAATGAAAGATTTAATGACCAAAATAGATTCAACAACGATATAC
CAAAGCCTAAACCAAGACTACAGCCTAATCAGCCACCGAAACAAGATAATAAATGCAGAGAA
GAGAATGGAGATTTTATCAATATTAGATTGTGTGCCTACGAGAAGGAATATTGCAATGACGG
ATATCTATCTCCTGCCTATTATATGTTAAAACAGGTGGATGATGAAGAAATGAGTTGCTGGT
CAGAACTATCGTCGTTGGTGAGATCCAGAAAGGCGGTGGGATTTCCTCTATTAAAGGCGGCT
AAACGTATTTCTCATGGATCGATGCTATATTTTGAACAGTTCAAAAACAGTAAAGTTGTGAA
ATTAACCCCGCAAGTTAAATGTTTAAATGATACTGTTATTTTTCAAACTGTAGTTATTTTAT
ATTCCATGTATAAACGTGGCATATATTCTAACGAATTTTGTTTTAATCTGGTTTCTATTCCC
AGAACGAACATTGTTTTTTCTGTTAATCAATTAATGTTTAACATTTGTACAGACATATTGGT
AGTTCTATCTATTTGCGGCAACCGGCTCTATAGAACAAATCTACCACAGTCGTGTTACTTAA
ATTTCATACACGGCCATGAGACAATAGCCCGTAGAGGATATGAACACTCCAATTACTTTTTC
GAGTGGTTGATAAAAAATCACATATCGCTATTGACCAAGCAAACGATGGATATTCTCAAGGT
AAAGAAAAAGTATGCTACAGGAGCACCAGTAAATAGGTTGTTAGAACCTGGTACACTGGTAT
ATGTGCCCAAAGAAGATTATTACTTTATAGGCATATCACTCACCGATGTGTCAATTAGCGAT
AATGTCAGAGTATTATTTTCCACAGATGGAATAGTGTTAGAAATAGAAGACTTTAATATCAA
GCATTTATTTATGGCAGGTGAGATGTTTGTTAGAAGTCAGTCTAGTACTATTATAGTATAA
1052 BP (SEQ ID NO:60)
Amino acid sequence:
MNTRTDVTNDNIDKNPTKRGDRNIPGRNERFNDQNRFNNDIPKPKPRLQPNQPPKQDNKCRE
ENGDFINIRLCAYEKEYCNDGYLSPAYYMLKQVDDEEMSCWSELSSLVRSRKAVGFPLLKAA
KRISHGSMLYFEQFKNSKVVKLTPQVKCLNDTVIFQTVVILYSMYKRGIYSNEFCFNLVSIP
RTNIVFSVNQLMFNICTDILVVLSICGNRLYRTNLPQSCYLNFIHGHETIARRGYEHSNYFF
EWLIKNHISLLTKQTMDILKVKKKYATGAPVNRLLEPGTLVYVPKEDYYFIGISLTDVSISD
NVRVLFSTDGIVLEIEDFNIKHLFMAGEMFVRSQSSTIIV (SEQ ID NO:81)
38.6 kDa
GenBank protein_id: AAR18069
Locus:VACAC2_001 chemokine-binding protein
5′ primer:
GAAGGAGATATACCATGCATCATCATCATCATCATATGCATGTGCCTGCC (SEQ ID NO:28)
3′ primer:
TGATGATGAGAACCCCCCCCTCAGACACACGCTTTGAGTT (SEQ ID NO:29)
Polynucleotide sequence:
ATGCATGTGCCTGCCAGTCTTCAGCAATCCTCCTCCTCGTGTACGGAAGAAGAAAACAAACA
TCATATGGGAATCGATGTTATTATCAAAGTCACAAAGCAAGACCAAACACCGACCAATGATA
AGATTTGCCAATCCGTAACGGAAATTACAGAGTCCGAGTCAGATCCAGATCCCGAGGTGGAA
TCAGAAGATGATTCCACATCAGTCGAGGATGTAGATCCTCCTACCACTTATTACTCCATCAT
CGGTGGAGGTCTGAGAATGAACTTTGGATTCACCAAATGTCCTCAGATTAAATCCATCTCAG
AATCCGCTGATGGAAACACAGTGAATGCTAGATTGTCCAGCGTGTCCCCAGGACAAGGTAAG
GACTCTCCCGCGATCACTCATGAAGAAGCTCTTGCTATGATCAAAGACTGTGAGGTGTCTAT
CGACATCAGATGTAGCGAAGAAGAGAAAGACAGCGACATCAAGACCCATCCAGTACTCGGGT
CTAACATCTCTCATAAGAAAGTGAGTTACGAAGATATCATCGGTTCAACGATCGTCGATACA
AAATGCGTCAAGAATCTAGAGTTTAGCGTTCGTATCGGAGACATGTGCAAGGAATCATCTGA
ACTTGAGGTCAAGGATGGATTCAAGTATGTCGACGGATCGGCATCTGAAGGTGCAACCGATG
ATACTTCACTCATCGATTCAACAAAACTCAAAGCGTGTGTCTGA 725 BP
(SEQ ID NO:61)
Amino acid sequence:
MHVPASLQQSSSSCTEEENKHHMGIDVIIKVTKQDQTPTNDKICQSVTEITESESDPDPEVE
SEDDSTSVEDVDPPTTYYSIIGGGLRMNFGFTKCPQIKSISESADGNTVNARLSSVSPGQGK
DSPAITHEEALAMIKDCEVSIDIRCSEEEKDSDIKTHPVLGSNISHKKVSYEDIIGSTIVDT
KCVKNLEFSVRIGDMCKESSELEVKDGFKYVDGSASEGATDDTSLIDSTKLKACV
(SEQ ID NO:82) 26.6 kDa
GenBank protein_id: AAR17912
Locus:VACAC2_139 nonessential glutaredoxin
5′ primer:
GAAGGAGATATACCATGCATCATCATCATCATCATATGGCCGAGGAATTT (SEQ ID NO:30)
3′ primer:
TGATGATGAGAACCCCCCCCTCAACAAGTTCTCAATACCC (SEQ ID NO: 31)
Polynucleotide sequence:
ATGGCCGAGGAATTTGTACAACAAAGGTTGGCCAATAACAAAGTGACAATTTTTGTCAAGTA
TACATGTCCTTTTTGTAGAAATGCACTGGATATTCTAAATAAGTTTAGTTTCAAAAGAGGAG
CGTATGAAATTGTCGATATTAAAGAATTTAAACCCGAAAATGAATTGCGGGATTATTTTGAA
CAAATTACTGGTGGTAGAACTGTTCCTAGAATCTTTTTTGGGAAAACTTCTATTGGTGGATA
TAGCGACCTGTTGGAAATAGACAACATGGACGCATTGGGTGATATTCTATCATCTATTGGGG
TATTGAGAACTTGTTGA 326 BP (SEQ ID NO:62)
Amino acid sequence:
MAEEFVQQRLANNKVTIFVKYTCPFCRNALDILNKFSFKRGAYEIVDIKEFKPENELRDYFE
QITGGRTVPRIFFGKTSIGGYSDLLEIDNMDALGDILSSIGVLRTC (SEQ ID NO:83)
12.0 kDa
GenBank protein_id: AAR17898
Locus:VACAC2_066 putative DNA-binding phosphoprotein
5′ primer:
GAAGGAGATATACCATGCATCATCATCATCATCATATGAATTCTCATTTT (SEQ ID NO:32)
3′ primer:
TGATGATGAGAACCCCCCCCTTAGTTCAAAAGTCTCGACG (SEQ ID NO:33)
Polynucleotide sequence:
ATGAATTCTCATTTTGCATCTGCTCATACTCCGTTTTATATCAATACCAAAGAAGGAAGATA
TCTGGTTCTAAAAGCCGTTAAAGTATGCGATGTTAGAACTGTAGAATGCGAAGGAAGTAAAG
CTTCCTGCGTACTCAAAGTAGATAAACCCTCATCACCCGCGTGTGAGAGAAGACCTTCGTCC
CCGTCCAGATGCGAGAGAATGAATAACCCTGGAAAACAAGTCCCGTTTATGAGAACGGACAT
GCTACAAAATATGTTCGCGGCTAATCGCGACAACGTGGCGTCGAGACTTTTGAACTAA
305 BP (SEQ ID NO:63)
Amino acid sequence:
MNSHFASAHTPFYINTKEGRYLVLKAVKVCDVRTVECEGSKASCVLKVDKPSSPACERRPSS
PSRCERMNNPGKQVPFMRTDMLQNMFAANRDNVASRLLN (SEQ ID NO:84) 11.2 kDa
GenBank protein_id: AAR18036
Locus:VACAC2_204 VACAC2_204
5′ primer:
GAAGGAGATATACCATGCATCATCATCATCATCATATGGATACAGATGTT (SEQ ID NO:34)
3′ primer:
TGATGATGAGAACCCCCCCCCTATATTCCTACATCGTGAC (SEQ ID NO:35)
Polynucleotide sequence:
ATGGATACAGATGTTACAAATGTAGAAGATATCATAAATGAAATAGATAGAGAGAAAGAAGA
AATACTAAAAAATGTAGAAATTGAAAATAATAAAAACATTAACAAGAATCATCCCAATGAAT
ATATTAGAGAAGCACTCGTTATTAATACCAGTAGTAATAGTGATTCCATTGATAAAGAAGTT
ATAGAATGTATCTGTCACGATGTAGGAATATAG 218 BP (SEQ ID NO:64)
Amino acid sequence:
MDTDVTNVEDIINEIDREKEEILKNVEIENNKNINKNHPNEYIREALVINTSSNSDSIDKEV
IECICHDVGI (SEQ ID NO:85) 8.0 kDa
GenBank protein_id: AAR17977
Locus:VACAC2_145 nonessential hydrophobic IV/IMV membrane
protein
5′ primer
GAAGGAGATATACCATGCATCATCATCATCATCATATGATAAGTAATTAC (SEQ ID NO:36)
3′ primer:
TGATGATGAGAACCCCCCCCTTAAATCGCCGAATGAACAA (SEQ ID NO:37)
Polynucleotide sequence:
ATGATAAGTAATTACGAGCCGTTGCTGCTGTTAGTTATAACATGCTGTGTACTACTATTTAA
TTTTACCATATCTTCGAAAACAAAAATAGATATTATTTTTGCAGTACAAACTATTGTTTTTA
TATGGTTTATATTCCACTTTGTTCATTCGGCGATTTAA 161 BP (SEQ ID NO:65)
Amino acid sequence:
MISNYEPLLLLVITCCVLLFNFTISSKTKIDIIFAVQTIVFIWFIFHFVHSAI
(SEQ ID NO:86) 5.9 kDa
GenBank protein_id: AAR17915
Locus:VACAC2_083 ssDNA-binding phosphoprotein
5′ primer:
GAAGGAGATATACCATGCATCATCATCATCATCATATGAGTAAGGTAATC (SEQ ID NO:38)
3′ primer:
TGATGATGAGAACCCCCCCCTTATACATTGAATATTGGCT (SEQ ID NO:39)
Polynucleotide sequence:
ATGAGTAAGGTAATCAAGAAGAGAGTTGAAACTTCACCAAGACCTACTGCATCTAGCGATTC
TCTACAGACTTGTGCGGGTGTTATAGAGTATGCAAAATCGATTAGTAAATCTAATGCAAAAT
GTATCGAATACGTTACACTAAATGCTTCTCAATACGCTAATTGTTCGTCTATCTCTATAAAA
CTTACTGATAGTTTATCTAGTCAAATGACTTCCACTTTTATTATGTTGGAAGGAGAGACTAA
ACTTTATAAAAATAAATCTAAACAAGATAGAAGCGATGGATACTTTCTAAAAATAAAAGTTA
CCGCGGCTAGTCCTATGTTGTATCAACTTCTAGAAGCCGTCTATGGAAACATTAAGCACAAG
GAACGCATTCCAAATTCTTTGCATAGTCTTTCGGTGGAAACTATTACAGAGAAAACATTTAA
GGATGAATCCATCTTCATCAACAAATTAAACGGATCCATGGTAGAATATGTTTCGACTGGAG
AATCATCCATTCTCAGATCTATAGAAGGTGAACTAGAATCACTCAGTAAAAGAGAAAGACAA
TTGGCCAAGGCAATTATCACACCTGTAGTTTTCTATAGATCCGGAACGGAAACAAAAATTAC
ATTCGCACTCAAGAAACTAATCATTGATAGAGAAGTGGTGGCTAACGTTATCGGACTCTCTG
GAGATAGTGAACGTGTATCAATGACTGAAAATGTAGAAGAAGATCTGGCTCGTAATCTGGGA
CTTGTTGATATTGATGATGAATATGATGAAGATAGCGATAAAGAAAAGCCAATATTCAATGT
ATAA 809 BP (SEQ ID NO:66)
Amino acid sequence:
MSKVIKKRVETSPRPTASSDSLQTCAGVIEYAKSISKSNAKCIEYVTLNASQYANCSSISIK
LTDSLSSQMTSTFIMLEGETKLYKNKSKQDRSDGYFLKIKVTAASPMLYQLLEAVYGNIKHK
ERIPNSLHSLSVETITEKTFKDESIFINKLNGSMVEYVSTGESSILRSIEGELESLSKRERQ
LAKAIITPVVFYRSGTETKITFALKKLIIDREVVANVIGLSGDSERVSMTENVEEDLARNLG
LVDIDDEYDEDSDKEKPIFNV (SEQ ID NO:87) 29.7 kDa
GenBank protein_id: AAR18010
Locus:VACAC2_108 profilin-like
5′ primer:
GAAGGAGATATACCATGCATCATCATCATCATCATATGGCTGAATGGCAT (SEQ ID NO:40)
3′ primer:
TGATGATGAGAACCCCCCCCTTAATTACCAGTTGCACGTA (SEQ ID NO:41)
Polynucleotide sequence:
ATGGCTGAATGGCATAAAATTATCGAGGATATCTCAAAAAATAATAAGTTCGAGGATGCCGC
CATCGTTGATTACAAGACTACAAAGAATGTTCTAGCTGCTATTCCTAACAGAACATTTGCCA
AGATTAATCCGGGTGAAATTATTCCTCTCAGCACTAATCGTAATATTCTAAAACCTCTTATT
GGTCAGAAATATTGTATTGTATATACTAACTCTCTAATGGACGAGAACACGTATGCTATGGA
GTTGCTTACTGGGTACGCCCCTGTATCTCCGATCGTTATAGCGAGAACTCATACCGCACTTA
TATTTTTGATGGGTAAGCCAACAACATCCAGACGTGACGTGTATAGAACGTGTAGAGATCAC
GCTACCCGTGTACGTGCAACTGGTAATTAA 401 BP (SEQ ID NO:67)
Amino acid sequence:
MAEWHKIIEDISKNNKFEDAAIVDYKTTKNVLAAIPNRTFAKINPGEIIPLITNRNILKPLI
GQKYCIVYTNSLMDENTYAMELLTGYAPVSPIVIARTHTALIFLMGKPTTSRRDVYRTCRDH
ATRVRATGN (SEQ ID NO:88) 14.7 kDa
GenBank protein_id: AAR17924
Locus:VACAC2_019 S-S bond formation pathway protein O:)
5′ primer:
GAAGGAGATATACCATGCATCATCATCATCATCATATGAAGAACGTATTG (SEQ ID NO:42)
3′ primer:
TGATGATGAGAACCCCCCCCTTATTCGGTAACAGGTGGCC (SEQ ID NO:43)
Polynucleotide sequence:
ATGAAGAACGTATTGATTATTTTCGGTAAACCGTATTGTAGTATTTGTGAAAATGTCAGTGA
GTCAGTAGAAGAATTAAAATCCGAGTATGATATACTCCATGTTGATATCTTATCATTTTTTT
TAAAGGATGGTGATTCAAGTATGCTGGGTGACGTAAAGCGCGGAACCCTAATAGGAAACTTT
GCAGCGCATCTATCTAACTACATCGTTTCCATTTTCAAATACAATCCACAGACAAAACAAAT
GGCATTTGTGGACATTAATAAATCCTTGGATTTCACCAAAACCGATAAATCGCTGGTGAATT
TGGAAATTCTAAAATCCGAAATAGAAAAAGCTACCTATGGAGTTTGGCCACCTGTTACCGAA
TAA 374 BP (SEQ ID NO:68)
Amino acid sequence:
MKNVLIIFGKPYCSICENVSESVEELKSEYDILHVDILSFFL
KDGDSSMLGDVKRGTLIGNFAAHLSNYIVSIFKYNPQTKQMAFVDINKSLDFTKTDKSLVNL
EILKSEIEKATYGVWPPVTE (SEQ ID NO:89) 13.7 kDa
GenBank protein_id: AAR17947
Locus:VACAC2_115 topoisomerase type IB
5′ primer:
GAAGGAGATATACCATGCATCATCATCATCATCATATGCGTGCACTTTTT (SEQ ID NO:44)
3′ primer:
TGATGATGAGAACCCCCCCCTCATCCATCCGTAGATGATT (SEQ ID NO:45)
Polynucleotide sequence:
ATGCGTGCACTTTTTTATAAAGATGGTAAACTCTTTACCGATAATAATTTTTTAAATCCTGT
ATTAGATGATAATCCAGCGTATGAGGTTTTGCAACATGTTAAAATTCCTACTCATTTAACAG
ATGTAGTAGTATATGAACAAACGTGGGAAGAGGCATTAACTAGATTAATTTTTGTGGGAAGC
GATTCAAAAGGACGTAGACAATACTTTTACGGAAAAATGCATGTACAGAATCGCAACGCTAA
AAGAGATCGTATTTTTGTTAGAGTATATAACGTTATGAAACGAATTAATTGTTTTATAAACA
AAAATATAAAGAAATCGTCCACAGATTCCAATTATCAGTTGGCGGTTTTTATGTTAATGGAA
ACTATGTTTTTTATTAGATTTGGTAAAATGAAATATCTTAAGGAGAATGAAACAGTAGGGTT
ATTAACACTAAAAAATAAACACATAGAAATAAGTCCCGATGAAATAGTTATCAAGTTTGTAG
GAAAGGACAAAGTTTCACATGAATTTGTTGTTCATAAGTCTAATAGACTATATAAACCGCTA
TTGAAACTGACGGATGATTCTAGTCCCGAAGAATTTCTGTTCAACAAACTAAGTGAACGAAA
GGTATACGAATGTATCAAACAGTTTGGTATTAGAATCAAGGATCTCCGAACGTATGGAGTCA
ATTATACGTTTTTATATAATTTTTGGACAAATGTAAAGTCCATATCTCCTCTTCCGTCACCA
AAAAAGTTAATAGCATTAACTATCAAACAAACTGCTGAAGTTGTAGGTCATACTCCATCAAT
TTCAAAAAGAGCTTATATGGCAACGACTATTTTAGAAATGGTAAAGGATAAAAATTTTTTAG
ATGTAGTATCTAAAACTACGTTCGATGAATTCCTATCTATAGTCGTAGATCACGTTAAATCA
TCTACGGATGGATGA 944 BP (SEQ ID NO:69)
Amino acid sequence:
MRALFYKDGKLFTDNNFLNPVLDDNPAYEVLQHVKIPTHLTDVVVYEQTWEEALTRLIFVGS
DSKGRRQYFYGKMHVQNRNAKRDRIFVRVYNVMKRINCFINKNIKKSSTDSNYQLAVFMLME
TMFFIRFGKMKYLKENETVGLLTLKNKHIEISPDEIVIKFVGKDKVSHEFVVHKSNRLYKPL
LKLTDDSSPEEFLFNKLSERKVYECIKQFGIRIKDLRTYGVNYTFLYNFWTNVKSISPLPSP
KKLIALTIKQTAEVVGHTPSISKRAYMATTILEMVKDKNFLDVVSKTTFDEFLSIVVDHVKS
STDG (SEQ ID NO:90) 34.6 kDa
GenBank protein_id: AAR17875
Locus:VACAC2_043 ankyrin-like
5′ primer:
GAAGGAGATATACCATGCATCATCATCATCATCATATGGATCTGTCACGA (SEQ ID NO:46)
3′ primer:
TGATGATGAGAACCCCCCCCTTAGTTTTTCTTTACACAAT (SEQ ID NO:47)
Polynucleotide sequence:
ATGGATCTGTCACGAATTAATACTTGGAAGTCTAAGCAGCTGAAAAGCTTTCTCTCTAGTAA
AGATACATTTAAGGCGGATGTCCATGGACATAGTGCCTTGTATTATGCAATAGCTGATAATA
ACGTGCGTCTAGTATGTACGTTGTTGAACGCTGGAGCATTGAAAAATCTTCTAGAGAATGAA
TTTCCATTACATCAGGCAGCCACATTGGAAGATACCAAAATAGTAAAGATTTTGCTATTCAG
TGGACTGGATGATTCACAATTTGATGACAAGGGAAACACCGCATTGTATTATGCGGTTGATA
GTGGTAACATGCAAACGGTAAAACTGTTTGTTAAGAAAAATTGGAGACTGATGTTCTATGGG
AAAACTGGATGGAAAACTTCATTTTATCATGCCGTCATGCTTAATGATGTAAGTATTGTTTC
CTACTTTCTTTCAGAGATACCATCTACTTTTGATCTGGCTATTCTCCTTAGTTGTATTCACA
TCACTATAAAAAATGGACACGTGGATATGATGATTCTCTTGCTCGACTATATGACGTCGACA
AACACCAATAATTCCCTTCTCTTCATTCCGGACATTAAATTGGCTATAGATAATAAAGACAT
TGAGATGTTACAGGCTCTGTTCAAATACGACATTAATATCTATTCTGCTAATCTGGAAAATG
TACTATTGGATGATGCCGAAATAGCTAAAATGATTATAGAAAAGCATGTTGAATACAAGTCT
GACTCCTATACAAAAGATCTCGATATAGTCAAGAATAATAAATTGGATGAAATAATTAGCAA
AAACAAGGAACTCAGACTCATGTACGTCAATTGTGTAAAGAAAAACTAA 854 BP
(SEQ ID NO:70)
Amino acid sequence:
MDLSRINTWKSKQLKSFLSSKDTFKADVHGHSALYYAIADNNVRLVCTLLNAGALKNLLENE
FPLHQAATLEDTKIVKILLFSGLDDSQFDDKGNTALYYAVDSGNMQTVKLFVKKNWRLMFYG
KTGWKTSFYHAVMLNDVSIVSYFLSEIPSTFDLAILLSCIHITIKNGHVDMMILLLDYMTST
NTNNSLLFIPDIKLAIDNKDIEMLQALFKYDINIYSANLENVLLDDAEIAKMIIEKHVEYKS
DSYTKDLDIVKNNKLDEIISKNKELRLMYVNCVKKN (SEQ ID NO:91) 31.3 kDa
GenBank protein_id: AAR18027
Locus:VACAC2_177 VACAC2_195
5′ primer:
GAAGGAGATATACCATGCATCATCATCATCATCATATGGCGATGTTTTAC (SEQ ID NO:48)
3′ primer:
TGATGATGAGAACCCCCCCCTTACCTCGATGGTGCCGCCG (SEQ ID NO:49)
Polynucleotide sequence:
ATGGCGATGTTTTACGCACACGCTCTCGGTGGGTACGACGAGAATCTTCATGCCTTTCCTGG
AATATCATCGACTGTTGCCAATGATGTCAGGAAATATTCTGTTGTGTCAGTTTATAATAACA
AGTATGACATTGTAAAAGACAAATATATGTGGTGTTACAGTCAGGTGAACAAGAGATATATT
GGAGCACTGCTGCCTATGTTTGAGTGCAATGAATATCTACAAATTGGAGATCCGATCCATGA
TCAAGAAGGAAATCAAATCTCTATCATCACATATCGCCACAAAAACTACTATGCTCTAAGCG
GAATCGGGTACGAGAGTCTAGACTTGTGTTTGGAAGGAGTAGGGATTCATCATCACGTACTT
GAAGCAGGAAACGCTGTATATGGAAAAGTTCAACATGATTATTCTACTATCAAAGAGAAGGC
CAAAGAAATGAGTACACTTAGTCCAGGACCTATAATTGATTACCACGTCTGGATAGGAGATT
GTATCTGTCAAGTTACTGCTGTGGACGTACATGGAAAGGAAATTATGAAAATGAGATTCAAA
AAGGGTGCGGTGCTTCCGATCCCAAATCTGGTAAAAGTTAAACTTGGGGAGAATGATACAGA
AAATCTTTCTTCTACTATATCGGCGGCACCATCGAGGTAA 659 BP (SEQ ID NO:71)
Amino acid sequence:
MAMFYAHALGGYDENLHAFPGISSTVANDVRKYSVVSVYNNKYDIVKDKYMWCYSQVNKRYI
GALLPMFECNEYLQIGDPIHDQEGNQISIITYRHKNYYALSGIGYESLDLCLEGVGIHHHVL
EAGNAVYGKVQHDYSTIKEKAKEMSTLSPGPIIDYHVWIGDCICQVTAVDVHGKEIMKMRFK
KGAVLPIPNLVKVKLGENDTENLSSTISAAPSR (SEQ ID NO:92) 24.2 kDa
GenBank protein_id: AAR18040
Locus:VACAC2_072 VACAC2_208
5′ primer:
GAAGGAGATATACCATGCATCATCATCATCATCATATGACGGCCAACTTT (SEQ ID NO:50)
3′ primer:
TGATGATGAGAACCCCCCCCTCAATTCATACGCCGGAATA (SEQ ID NO:51)
Polynucleotide sequence:
ATGACGGCCAACTTTAGTACCCACGTCTTTTCACCACAGCACTGTGGATGTGACAGACTGAC
CAGTATTGATGACGTCAAACAATGTTTGACTGAATATATTTATTGGTCGTCCTATGCATACC
GCAACAGGCAATGCGCTGGACAGTTGTATTCCACACTCCTCTCTTTTAGAGATGATGCGGAA
TTAGTGTTCATCGACATTCGCGAGCTGGTAAAACATATGCCGTGGGATGATGTCAAAGATTG
TGCAGAAATCATCCGTTGTTATATACCGGATGAGCAAAAAACCATCAGAGAGATTTCGGCCA
TCATCGGACTTTGTGCATATGCTGCTACTTACTGGGGAGGTGAAGACCATCCCACTAGTAAC
AGTCTGAACGCATTGTTTGTGATGCTTGAGATGCTAAATTACGTGGATTATAACATCATATT
CCGGCGTATGAATTGA 449 BP (SEQ ID NO:72)
Amino acid sequence:
MTANFSTHVFSPQHCGCDRLTSIDDVKQCLTEYIYWSSYAYRNRQCAGQLYSTLLSFRDDAE
LVFIDIRELVKHMPWDDVKDCAEIIRCYIPDEQKTIREISAIIGLCAYAATYWGGEDHPTSN
SLNALFVMLEMLNYVDYNIIFRRMN (SEQ ID NO:93) 16.5 kDa

The PCR reactions contained 100 ng vaccinia virus genomic DNA, 25 nM final concentration of 5′ and 3′ primers. Polymerase, PCR buffer and nucleotides were from Clontech. The reaction temperature and times for the first PCR reaction were: 94° C. for 2 minutes, followed by 30 cycles of: 94° C. for 30 seconds, 48° C. for 1 min., and 68° C. for 2.5 minutes.

Following the first PCR reaction, an aliquot of each PCR reaction containing 100 ng of PCR product from the previous step was transferred into a PCR reaction containing the the TAP promoter and terminator primers. The sequences of these primers was:

His Promoter Primer:

(SEQ ID NO:6)

5′CGGTCACGCTTGGGACTGCCATAGGCTGGCCCGGTGATGCCGGCCACG
ATGCGTCCGGCGTAGAGGATCGAGATCTCGATCCCGCGAAATTAATACGA
CTCACTATAGGGAGACCACAACGGTTTCCCTCTAGAAATAATTTTGTTTA
ACTTTAAGAAGGAGATATACC 3′

His Terminator Primer:

(SEQ ID NO:7)

5′GGGGGGGGTTCTCATCATCATCATCATCATTAATAAAAGGGCGAATTC
CAGCACACTGGCGGCCGTTACTAGTGGATCCGGCTGCTAACAAAGCCCGA
AAGGAAGCTGAGTTGGCTGCTGCCACCGCTGAGCAATAACTAGCATAACC
CCTTGGGGCCTCTAAACGGGTCTTGAGGGGTTTTTTGCTGAAAGGAGGAA
CTATATCCGGAGCGACTCCCACGGCACGTTGGCAAGCTCG 3′

The reaction temperature and times for the second PCR reaction were: 94° C. for 2 minutes, followed by 30 cycles of: 94° C. for 30 seconds, 48° C. for 60 seconds, and 68° C. for 2.5 minutes.

Protein Expression

The TAP fragments generated by PCR were used as templates for in vitro protein expression using a Roche RTS100 transcription/translation kit according to manufacturer's instructions. Approximately 0.5˜1.0 μg PCR product was used as template, producing approximately 0.5˜5.0 μg of protein per template.

Protein Purification

MagneHis nickel-coated magnetic beads (Promega) were used to purify the expressed proteins. 15 μl of Ni-magnetic beads (Promega) were pipetted into each well of a microtiter plate. To each well 50 μl wash buffer (50 mM NaHPO₄, pH8.0, 300 mM NaCl, 100 mM imidazole) was added with mixing and the plates were placed on a magnetic stand. The supernatant was removed and wash was repeated. 50 μl of the Protein mixture was added with gentle pipetting. The mixture was incubated at room temperature for 2 minutes. The beads were then separated using a magnetice stand, washed 3 times with 150 μl wash buffer and the bound protein was eluted from the beads with 50 μl of 50 mM NaHPO₄, pH 8.0, 300 mM NaCl, 250 mM imidazole.

Western Blot

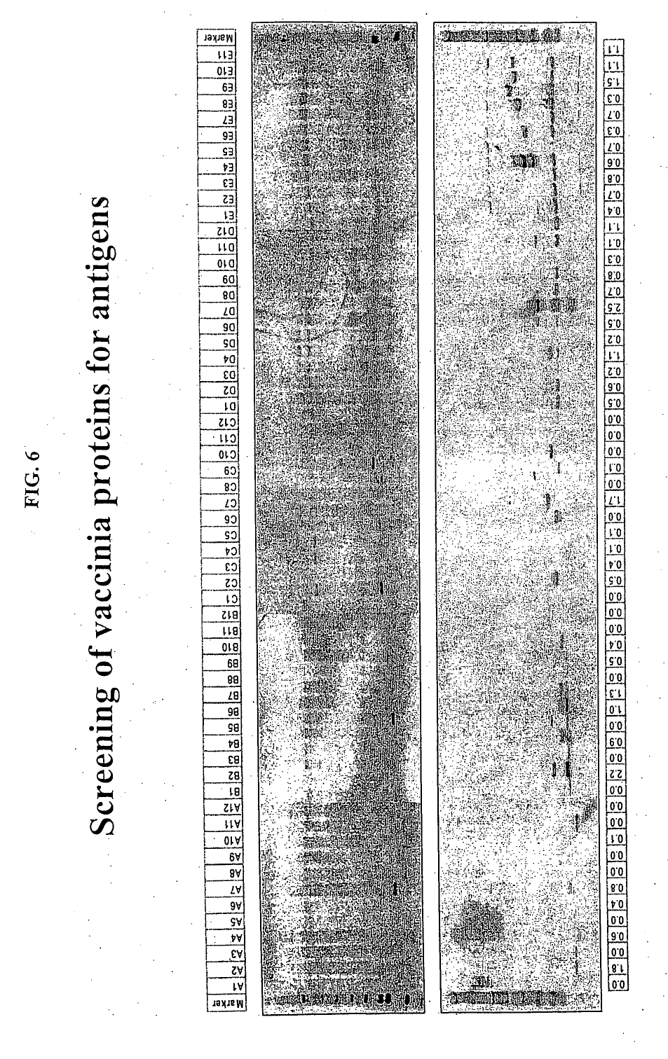

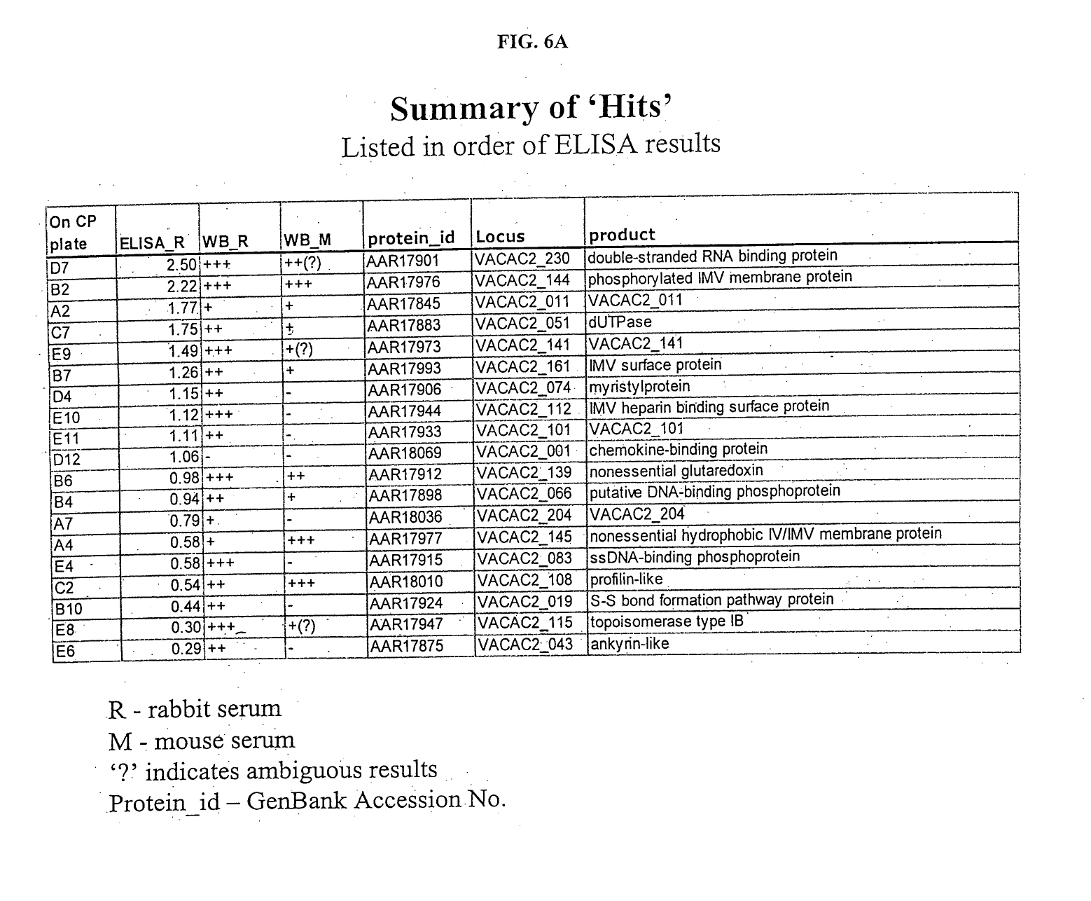

15 μl of the purified proteins were resolved on 4-12% SDS-polyacrylamide gels and transferred to nitrocellulose membranes The membranese were blocked in TBST/1% BSA, followed by incubation with TBST/1% BSA containing 500-fold diluted rabbit anti-vaccinia virus serum. The blots were washed and then incubated with alkaline phosphotase-conjugated goat-anti rabbit serum secondary antibody. Colorimetric development was used to develop the blots. The results of this analysis are shown in FIG. 6.

ELISA

The wells of Nunc-Immuno MaxiSorp 96-well plates were coated with with 5 μl expressed protein diluted in 95 μl PBS. The plates were mixed well on a shaker and then incubated overnight at 4° C. The plates were washed with PBS+0.05% Tween-20 for 5 min. with shaking at 200 rpm. 200 μl PBS+1% BSA blocking solution was added and the plates incubated for 1 hr at room temperature, with shaking at 150 rpm. The blocking buffer was removed and 100 μl primary antibody (rabbit anti-vaccinia virus polyclonal antibodies diluted 1:500 in blocking solution) was added. Following 1 hr incubation at room temperature with shaking at 150 rpm, the plates were washed 3 times with PBS+0.05% Tween-20. 100 μl second antibody(Anti-Rabbit IgG(H+L)-HRP conjugated, (Promega) diluted 1:2500 in blocking solution was added to each well and the plates were incubated for 1 hr at room temperature, with shaking at 150 rpm. After washing 3 times, 100 μl TMB substrate solution (Promega) was added to each well and the blue color was allowed to develop for 15 min at room temperature without shaking. 100 μl 1N HCl was added to each well to stop the reaction and change the blue color to yellow. The plates were read at 450 nm after 30 min. The results of this analysis are shown in FIG. 6.

### Example 3

### Using the Vaccinia Virus Proteome to Identify the Antigenic Targets of Cell-Mediated Immunity in Vaccinia Virus Vaccinated Mice and Humans

The following is a method that is used to systematically screen and identify antigens in vaccinia virus that give rise to a protective cell-mediated immune response. Through the use of TAP technology coding sequences of the vaccinia virus genome are amplified. The PCR reactions are performed such that each amplified coding sequence becomes transcriptionally active. The resulting TAP fragments are expressed to produce vaccinia virus polypeptides. Each of the polypeptides are delivered into dendritic cells, located in 96-well plates, using a polypeptide delivery reagent. Serum from vaccinia virus immunized humans is added to each of the different wells.

An IFN-γ ELISPOT assay is run using the following materials and method:

Materials:

Millipore 96-well multi-screen filtration plates (Millipore #MAIP S45-10) (Millipore, Bedford, Mass.)

Anti-IFN-g purified MAb (Clone 1-DIK) (MABTECH #3420-3) (Mabtech, Naka, Sweden)

Anti-IFN-g Biotinylated MAb (Clone 7-B6-1) (MABTECH #3420-6) (Mabtech, Naka, Sweden)

Streptavidin-Alkaline Phosphatase (MABTECH #3310-8) (Mabtech, Naka, Sweden)

Alkaline Phosphate Substrate Kit (BIO-RAD #170-6432) (Bio-Rad, Hercules, Calif.)

Carbonate Buffer pH 9.6 (0.2 μM sterile filtered)

RPMI-1640 Medium (GIBCO #22400-089) (Gibco, Grand Island, N.Y.)

Fetal Bovine Serum (Sigma #F4135-500 mL) (Sigma, St. Louis, Mo.)

1×PBS (Prepared from 10×PBS DIGENE #3400-1010) (DIGENE, Gaithersburg, Md.)

Tween® 20 (J.T. Baker #X251-07) (J.T. Baker, Phillipsburg, N.J.)

Method:

96-well plates are coated with Coating Antibody (anti-IFN-g Clone 1-DIK) at 10-15 μg/mL (100 μL/well) and incubated at 4° C. overnight. Using aseptic technique, plates are flicked to remove Coating Antibody and washed 6 times with RPMI-1640. Plates are blocked with 100 μL/well of RPMI-1640+10% FBS (or Human AB serum) for 1-2 hours at room temperature. Plates are flicked to remove blocking buffer and 100 μL/well of antigen specific or control peptides are added at a final concentration of 10 μg/well. Peripheral blood lymphocytes (PBL) are added at 4×105/well and 1×105/well. Plates are incubated at 37° C./5% CO₂ for 36 hours. Plates are flicked to remove cells and washed 6 times with PBS+0.05% Tween® 20 at 200-250 μL/well. Plates are blot dried on paper towels.

Biotinylated antibody (anti-IFN-g Clone 7-B6-1) diluted 1:1,000 in 1×PBS at 100 μL/well is added. The resulting solution is incubated for 3 hours at room temperature. Plates are flicked to remove biotinylated antibody and washed 6 times with PBS+0.05% Tween® 20 at 200-250 μL/well. Plates are blot dried on paper towels. Streptavidin alkaline phosphatase is added at 100 μL/well diluted 1:1,000 in 1×PBS. The plates are incubated for 1 hour at room temperature. Plates are flicked to remove the streptavidin alkaline phosphatase and washed 6 times with 0.05% Tween® 20 at 200-250 μL/well. The plates are washed again 3 times with 1×PBS at 200-250 μL/well. The plates are blot dried on paper towels.

Substrate is added at 100 μL/well for 10-15 minutes at room temperature. The substrate is prepared according to manufacturer's protocol. The 25× substrate buffer is diluted in dH20 to a 1× concentration. Reagent A & B are each diluted 1:100 in the 1× substrate buffer. Rinsing plates with generous amounts of tap water (flooding plate and flicking several times) stops calorimetric substrate. Plates are allowed to dry overnight at room temperature in the dark. Spots corresponding to IFN-γ producing cells are determined visually using a stereomicroscope (Zeiss KS ELIspot). Results can be expressed as the number of IFN-γ-secreting cells per 10⁶ spleen cells. Responses are considered positive if the response to test Vaccinia peptide epitope is significantly different (p<0.05) as compared with the response to no peptide and if the stimulation index (SI=response with test peptide/response with control peptide) is greater than 2.0.

### Example 4

### Cellular Vaccine Antigen Screen

A human volunteer was immunized with irradiated sporozoites from P. falciparum, the infectious agent responsible for malaria. Dendritic cells from the volunteer were isolated and cultured. Recombinant CSP polypeptide from P. falciparum was delivered to dendritic cells with or without polypeptide delivery reagents described in U.S. patent application Ser. No. 09/738,046, entitled “Intracellular Protein Delivery Reagent,” which is hereby incorporated by reference in its entirety. T-cells isolated from the immunized volunteer were added to the cultures. The EliSpot assay identified 120 CSP antigen specific T-cells out of 250,000 T-cells that were added to the culture when CSP was added to the culture together with said delivery reagents. When CSP was added without said delivery reagents, the signal was barely above background.

### Example 5

### DNA Immunization of Mice

Experiments are set up with five animals per group, consisting of four week old BALB/c female mice, averaging 40 animals per experiment. These mice are immunized IM in each tibialis anterior muscle with 50 μg plasmid DNA or transcriptionally active PCR fragment encoding selected vaccinia virus antigens, 3 times at 3 week intervals.

Sera is collected 10 days after each immunization for antibody studies. Blood samples (˜50 ul) are collected from the mice by orbital bleed with a sterilized pasture pipette. The mice are bled about once a week at a volume of approximately 50 μl.

Splenocytes are harvested at 14 days after the 3rd immunization and pooled for T-cell studies such as IFN-γ ELIspot assays. Tissue collections are done on animals euthanized via CO₂ (SOP 98.19) at the end of the experiment. The experiments can be five animals/group, averaging 40 animals/experiment×4 experiments for a total of 160 mice.

### Example 6

### Preparation of Vaccinia Virus Genomic DNA to be Used as PCR Template

Crude-stock Vaccinia virus is used to infect 10 liters of HeLa cell spinner culture. 2-3 days after infection, cells are harvested by centrifugation and broken by ounce in 10 mM Tris-HCl (pH 9.0) on ice. Nuclei is pelleted, washed and re-pelleted after which supernatants are combined, trypsinized, and layered onto a 36% sucrose cushion. After centrifugation, the pelleted viruses are dispersed and trypsinized, and overlaid onto a continuous 5-40% sucrose gradient. Bands are collected, diluted and the virus is washed. A deoxycholate extract of the purified virions are centrifuged to remove debris, the pellet re-extracted, and the combined supernatants applied to a DEAE cellulose column, equilibrated in 0.1 M KCl., Tris-HCl pH 8.4. After washing the column with 250 mM KCl, Vaccinia DNA is eluted with 0.7 M KCl, ethanol precipitated and redissolved in TE buffer.

### Example 7

### Preparation of Human Dendritic Cells

Dendritic cells were ordered from Allcells: Cat # PB002 (NPB-Mononuclear Cells). The cells were in 50 mL buffer. The cells were counted immediately, the total number was 312.5×10⁶. The cells were pelleted, and resuspended in 25 mL RPMI-1640 containing DNAse. This solution (30 μg/mL) was incubated for 5 minutes at room temperature. The cells were washed twice with complete medium. The cells were resuspended at 10×10⁶ cells/3 mL. Twelve 10 mm dishes containing 10 mL complete medium in each dish were used. The cells were incubated at 37° C. for 3 hours. The non-adherent cells were removed by gently shaking plates and aspirating supernatant. Afterwards, the dishes containing adherent cells were washed 3 times with 10 mL of RPMI-1640 containing 2% Human Serum. 10 mL of culture medium were added to each plate containing 50 ng/mL GM-CSF and 500 u/mL IL-4. This culture medium was added until day 4. After day 4, culture medium without GM-CSF and IL-4 was added. The transfection was done on day 5. The complete medium consisted of RPMI-1640 (455 mL), 5% Human AB Serum (25 mL), Non-essential Amino Acids (5 mL), Sodium Pyruvate (5 mL), L-Glutamine (5 mL), and Penicillin-Streptomycin (5 mL).

### Example 8

### Generation of Dendritic Cells from Mouse Bone Marrow

Cells were taken from the bones of one mouse (2 femur and 2 tibiae without removing the macrophages). The red blood cells were obtained from the bone marrow and lysed. The cells were counted (51×10⁶ cells, total) and cultured in a growth medium (2.5×10⁶ cells/plate, 10 mL/plate) for 8 days before transfection. On day 4 another 10 mL of growth medium was added. On day 6, 10 mL of the old medium was taken from each plate and the cells were pelleted. The cells were resuspended in 10 mL medium with 10 ng/mL GM-CSF and 2.5 ng/mL IL-4. The cells were placed back into the culture. The cells were cultured until transfection on day 8. On the day of transfection, 2.5×10⁶ cells were harvested from each dish. The growth medium for mbmDC contained DMEM/Iscove, 10% FCS, 50 uM β-mercaptoethanol, 1×Penicillin/Streptomycin, 2 mM L-Glutamine, 10 mM Hepes, 1×Non-essential amino acids, 20 ng/mL rmGM-CSF, and 5 ng/mL rmIL-4.

### Example 9

### Adding an HA Epitope Tag

Oligos are designed using TAP promoter and terminator fragments from pCMVm and pTP-SV40, respectively, and adding the nucleotide sequence encoding the HA epitope tag. For adding the HA epitope to the 5′ end of the coding sequence the following sequences is used:

| | | |
|---|---|---|
| Promoter 5′: | CCGCCATGTTGACATTG | (SEQ ID NO:2) |
| Promoter 3′: | GGCAGATCTGGGAGGCTAGCGTAA | (SEQ ID NO:3) |
| | TCCGGAACATCGTATGGGTACATT | |
| | GTTAAGTCGACGGTGC | |

For adding the HA epitope to the 3′ end of the coding sequence, the following sequences is used:

| | | |
|---|---|---|
| Terminator 5′: | GATCCCGGGTACCCATACGAT | (SEQ ID NO:4) |
| | GTTCCGGATTACGCTTAGGGG | |
| | AGATCTCAGACATG | |
| Terminator 3′: | CAGGATATCATGCCTGCAGGA | (SEQ ID NO:5) |
| | CGACTCTAGAG | |

The Method Includes:

PCR is used to amplify a new HA-promoter utilizing pCMVm as a template and a new HA-terminator utilizing pTP-SV40 as a template. The resulting PCR products are gel purified using QIAGEN QIAquick Gel Extraction Kit (Qiagen, Seattle, Wash.). The PCR products and both plasmids (pCMVm & pTP-SV40) are digested with EcoRV and BglII restriction enzymes. All digested products are gel purified using QIAquick Gel Extraction Kit. The HA-promoter and HA-terminator are ligated separately into the digested pCMVm and pTP-SV40 plasmids. These plasmids are transformed into DH5, grown overnight on LB plates containing Kanamycin, colonies are selected and grown in LB media containing Kanamycin. QIAGEN QIAprep Spin Miniprep Kit is used to isolate plasmids. Plasmids are digested using EcoRV and BglII. Digests are run on a gel to identify clones containing plasmid with insert of correct size. The plasmids are sequenced to confirm inserts are correct. A prep culture is grown, plasmids are isolated, plasmids are digested with EcoRV and BglII, and promoter and terminator fragments are gel purified. Epi-TAP-5 HA and Epi-TAP-3 HA kits are used.

What is claimed is:

1. An isolated polynucleotide encoding a vaccinia virus polypeptide that is antigenic in a mammal, wherein the polynucleotide is selected from the group consisting of:
(a) SEQ ID NOS: 52-72; or
(b) a fragment thereof, wherein the fragment encodes an antigenic polypeptide.

2. The isolated polynucleotide of claim 1, wherein said mammal is a rabbit, human or mouse.

3. An isolated polynucleotide encoding a highly immunogenic vaccinia virus antigen, wherein the polynucleotide is selected from the group consisting of:
(a) SEQ ID NOS: 52-72; or
(b) a fragment thereof, wherein the fragment encodes an immunogenic polypeptide.

4. The polynucleotide of claim 1, wherein said highly immunogenic vaccinia virus antigen reacts with polyclonal antibodies from at least two different species directed to vaccinia virus.

5. The polynucleotide of claim 1, wherein said highly immunogenic vaccinia virus antigen is detected by both ELISA and Western blotting using a polyclonal antibody that is directed to vaccinia virus.

6. A TAP polynucleotide comprising:
(a) a 5′ TAP polynucleotide sequence;
(b) a vaccinia virus polynucleotide sequence; and
(c) a 3′TAP polynucleotide sequence.

7. The TAP polynucleotide of claim 6, wherein the vaccinia virus polynucleotide sequence is selected from the group consisting of: SEQ ID NOS: 52-72.

8. The polynucleotide of claim 6, wherein the 5′ TAP polynucleotide sequence comprises a promoter.

9. The polynucleotide of claim 6, wherein the 5′TAP polynucleotide sequence is selected from the group consisting of: SEQ ID NOS: 2, 3, 6, and 8.

10. The polynucleotide of claim 6, wherein the 3′ TAP polynucleotide sequence comprises a terminator.

11. The polynucleotide of claim 6, wherein the 3′TAP polynucleotide sequence is selected from the group consisting of: SEQ ID NOS:4, 5, 7, and 9.

12. A primer pair for amplifying a vaccinia virus polynucleotide selected from the group consisiting of: SEQ ID NOS: 10 and 11; 12 and 13; 14 and 15; 16 and 17; 18 and 19; 20 and 21; 22 and 23; 24 and 25; 26 and 27; 28 and 29; 30 and 31; 32 and 33; 34 and 35; 36 and 37; 38 and 39; 40 and 41; 42 and 43; 44 and 45; 46 and 47; 48 and 49; 50 and 51.

13. An isolated vaccinia virus peptide or polypeptide that is antigenic, wherein said peptide or polypeptide is selected from:
(a) SEQ ID NOS: 73-93; or
(b) a fragment thereof, wherein the fragment is antigenic.

14. An isolated vaccinia virus peptide or polypeptide that is highly immunogenic, wherein the peptide or polypeptide is selected from the group consisting of:
(a) SEQ ID NOS: 73-93; or
(b) a fragment thereof, wherein the fragment is highly immunogenic.

15. The peptide or polypeptide of claim 14, wherein said peptide or polypeptide reacts with polyclonal antibodies from at least two different species that are directed to vaccinia virus.

16. The peptide or polypeptide of claim 14, wherein said peptide or polypeptide is detected by both ELISA and Western blotting using a polyclonal antibody that is directed to vaccinia virus.

17. A recombinant vaccinia virus peptide or polypeptide, wherein the amino terminus of said peptide or polypeptide comprises a HA tag or a His tag, and the carboxy terminus of said peptide or polypeptide is selected from the group consisting of: SEQ ID NOS: 73-93.

18. A recombinant vaccinia virus peptide or polypeptide, wherein the carboxy terminus of said peptide or polypeptide comprises a HA tag or a His tag, and the amino terminus of said peptide or polypeptide is selected from the group consisting of: SEQ ID NOS: 73-93.

19. A recombinant vaccinia virus peptide or polypeptide selected from the group consisting of: SEQ ID NOS: 73-93, wherein said peptide or polypeptide is expressed in an in vitro transcription-translation system.

20. The recombinant vaccinia virus peptide or polypeptide of claim 19, wherein said in vitro transcription-translation system is a T7 polymerase system.

21. An immunogenic composition for inducing an immunological response in a mammalian host against vaccinia virus, comprising a nucleic acid that encodes and expresses in vivo in a mammalian host cell at least one immunogenic peptide or polypeptide selected from the group consisting of:
(a) SEQ ID NOS: 73-93; or
(b) a fragment thereof, wherein the fragment is immunogenic.

22. The immunogenic composition of claim 21, wherein said nucleic acid comprises a plasmid.

23. The immunogenic composition of claim 21, wherein said nucleic acid comprises a TAP fragment.

24. The immunogenic composition of claim 21, wherein said nucleic acid induces a humoral immune response.

25. The immunogenic composition of claim 21, wherein said nucleic acid induces a cell-mediated immune response.

26. The immunogenic composition of claim 21, further comprising an adjuvant.

27. An immunogenic composition comprising at least one isolated vaccinia virus peptide or polypeptide selected from the group consisting of:
(a) SEQ ID NOS: 73-93; or
(b) a fragment thereof, wherein the fragment is immunogenic.

28. The immunogenic composition of claim 27, wherein said isolated vaccinia virus peptide or polypeptide is expressed in an in vitro transcription-translation system.

29. The immunogenic composition of claim 27, further comprising an adjuvant.

30. A method of generating an immune response in a mammalian host against vaccinia comprising: administering to said mammalian host an immunogenic composition comprising at least one nucleic acid selected from the group of SEQ ID NO: 52-72, fragments thereof or combinations thereof, wherein said nucleic acid encodes and expresses in vivo at least one immunogenic peptide or polypeptide, whereby said immune response against vaccinia is generated.

31. A method of generating an immune response in a mammalian host against vaccinia comprising: administering to said mammalian host an immunogenic composition comprising at least one nucleic acid encoding and expressing in vivo at least one immunogenic peptide or polypeptide selected from the group of SEQ ID NO: 73-93, fragments thereof or combinations thereof, whereby said immune response against vaccinia is generated.

32. A method of generating an immune response in a mammalian host against vaccinia comprising administering to said mammalian host an immunogenic composition comprising at least one immunogenic peptide or polypeptide selected from SEQ ID NO: 73-93, fragment thereof or combinations thereof, whereby said immune response against vaccinia is generated.

33. The method according to claims 30, 31 or 32, wherein said immunogenic composition further comprises and adjuvant.

34. A kit comprising:
a) at least one vaccinia immunogenic composition selected from:
i) a nucleic acid selected from the group consisting of SEQ ID NO: 52-72, f fragments thereof or combinations thereof; or
ii) a peptide selected from the group consisting of SEQ ID NO: 73-93, fragments thereof or combinations thereof; and
b) an adjuvant.

35. The kit of claims 34, wherein when said vaccinia immunogenic composition is a nucleic acid the kit further comprises an expression system.

36. The kit of claim 34 or 35 comprising at least two of said immunogenic compositions.

37. The kit of claim 36 comprising at least two of said peptides.

38. The kit of claim 36, comprising at least two of said nucleic acids.
